SEQUENCE LISTING (31 sequences)

SEQ ID NO: 1 - DNA, 1566 bp, Homo sapiens
gcggcggcagcggaggcggcggctccagccggcgcggcgcgaggctcggcggtgggatccggcgggcggtgctagctccgcgctccctgcctcgctcgctgccgggggcggtcggaaggcgcggcgcgaagcccgggtggcccgagggcgcgactctagccttgtcacctcatcttgcccccttggttttggaagtcctgaagagttggtctggaggaggaggaggacattgatgtgcttggtgtgtggccagtggtgaagagatggctgctcctgtcccgtgggcctgctgtgctgtgcttgccgccgccgccgcagttgtctacgcccagagacacagtccacaggaggcaccccatgtgcagtacgagcgcctgggctctgacgtgacactgccatgtgggacagcaaactgggatgctgcggtgacgtggcgggtaaatgggacagacctggcccctgacctgctcaacggctctcagctggtgctccatggcctggaactgggccacagtggcctctacgcctgcttccaccgtgactcctggcacctgcgccaccaagtcctgctgcatgtgggcttgccgccgcgggagcctgtgctcagctgccgctccaacacttaccccaagggcttctactgcagctggcatctgcccacccccacctacattcccaacaccttcaatgtgactgtgctgcatggctccaaaattatggtctgtgagaaggacccagccctcaagaaccgctgccacattcgctacatgcacctgttctccaccatcaagtacaaggtctccataagtgtcagcaatgccctgggccacaatgccacagctatcacctttgacgagttcaccattgtgaagcctgatcctccagaaaatgtggtagcccggccagtgcccagcaaccctcgccggctggaggtgacgtggcagaccccctcgacctggcctgaccctgagtcttttcctctcaagttctttctgcgctaccgacccctcatcctggaccagtggcagcatgtggagctgtccgacggcacagcacacaccatcacagatgcctacgccgggaaggagtacattatccaggtggcagccaaggacaatgagattgggacatggagtgactggagcgtagccgcccacgctacgccctggactgaggaaccgcgacacctcaccacggaggcccaggctgcggagaccacgaccagcaccaccagctccctggcacccccacctaccacgaagatctgtgaccctggggagctgggcagcggcgggggaccctcggcacccttcttggtcagcgtccccatcactctggccctggctgccgctgccgccactgccagcagtctcttgatctgagcccggcaccccatgaggacatgcagagcacctgcagaggagcaggaggccggagctgagcctgcagaccccggtttctattttgcacacgggcaggaggaccttttgcattctcttcagacacaatttgtggagaccccggcgggcccgggcctgccgccccccagccctgccgcaccaagct

SEQ ID NO: 2 - protein, 372 aa, Homo sapiens
MAAPVPWACCAVLAAAAAVVYAQRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSGGGPSAPFLVSVPITLALAAAAATASSLLI

SEQ ID NO: 3 - DNA, 65516 bp, Homo sapiens
ctcctcctcaaaccaactagaaggggcgtccaaccagaaggcacccaaagccagccctcagggaccttttctctgctctcagaaaagcttagtgtataccaggctaacctcttcatattgaggcatcccaaattctagtcttaataatccctttttgcagaacctagggagttgaagttagacaatgggaacaattttgcaatagtgaaaaagaaagggaggaggtagatgtagggcaggagtgcccgtcagctagcaggtgctcaataaatatgtggagaaaaacagatctcaaagcctggaccccaccagagggaggtgttggctggccagatctgtaacaggggcagtacaggctacacaccagggtagcaggaagtgacaaggtcagataaggaattctgtggcctggaagctctctccttgcttggatgtctctctttctctacgttgcagtccttgtctcttagcaaaaggaccagaagctgcacttttgtaagactgatgggaaggccctccccctgcacagctgtccagagatttcaggattcctagattccccaagtgctggtgtggagatttgggcctgcctctgtctctgtctgtctctctccctggctttttggttagagacagaggggttcaaatacctactctactacctactggctttgtggcctcaaaaagtgacttaaggtctcaattacctcagaagtaaaatgggatcataacagtgcctatttcctaggactgtggtgaagattaagttaaatgagataatccagtcaagtgcttagtgctagccctagcaggtgctaaagcccccttcgtctgctggctttggcttcctcccagttgatgtctatgtctcctctgtcacggcctttctccccattttctctatcaacattatcctctgtctctgtcttttactgtctttctcttgatctctttggctctccttgtatctggccctgcctgcatgaccctgctccaaccctctggcacaattggcctaggtgaggtagggtaggggtgtccaggaaggtaaatttaaatatggcattcatggcagcagggtcatgcctgagcctgaacataggtgaaccacaatcgttggggtgagtacttctgtctatggccctttctgactgggcagaagagctgggggctcactctggcagtcctcttggaggaggggggacttgtcctgactgttctgggccgcccccacacctggacccaggccagatccccatagctagtgcctgcctggaggtgaggcaacctgcccaaaggccagaactgaaaggggtagccctctacctccacacagagttgcttacctgacctcaaaacattctggcccaccctcatccaagttctgctctgagcctcccataccccatcccaaaccctgtgtggaagcacattgcctccatagatctagtttctcttgtctctggctcttcctatctctgtctctatctatgtgtctctgtcttctcaatctcgatctatgactttggcaactgcataagccttgcacccctaagctaagaccaccagggataagaaactgcaggggttgaagagtctgtgttcttcacctggtccaaaaagctccttcttggtggtggtggtggggcgaacacaagaaatccagatgtcatttccatgtcactgtggacctgtgaaggcagggggcatggcatagaataggtgctcattaaatataaaccaaatgaaggaggggcaaacagaggactccaaacaaggaactgaatggctgaggctgtagcaggcaggaacatggagagactatggaaaggctgctgctcaactttgtgactcaacaaggagtcttctttgggggtcaggattggtctcaactagccctagatgctgccacacctgggccctggccagccccttcacgtcagctcttgggcagacagccagatgggtgagccactaagacttccccatcacttccctactcccaacacaccattgcctcaaggtgcccaccagcttcaagcagagtgctgggaatacagagccagagagggagaaactctgggcaggggcacacggcaggcagaggctgagacaggagtctggtgtcctggtagccagcctgggcctgtcatgaggatcctatgccagtacagtgatggggccagacccgaaacaggattttatgtggggagagaagccaggaatccttgaacatgtgggcagctgggcaaggggtggaacgtcccatcaggtaaggacagatgtgcagcttcttgtccttcttgttagataaagacaagcattgagacaaaaggttcagagagatagaatcagagcaaaagagagaagagagagcctgggagacagaccgagaaatagaaactctcagagacacagtcaggcacaaaaacaaagatgcagagaaagagatgtgagtcagggacacagacagggaaatgcagaagagagagccatggaattaaagacacagagaccaagatagggagggatatgaaatcagagagaaactgtctcagtcacagagcaacaatgtggagacagactcagacagagaaacacagagacagggagaaaaaaaaattgagagatagagagatggagaagagagagaggaggacagcgacagcaagatcaagagaggcagagagatggacagaggacaaggcagaaggaagcaaagacagggctaggcaaagaaagagatcaaaacattaggagtgggaaaagtgggcgagggacaaaggagtttcgtggcctcagtctctctagcctggtctccccagggtgttggacccttactttgctattctctggtttccatagcaacagggagggcagcgccaaatttgcatacacatttacatcctttctatttgaataattcaacaggtttctgtgcatattcatgagataatgaatgaaggtgactctcaatccctgctcaaaacctggctatggtatcccctgctttgtagtgtatggtatggctctgagcctggttttgtaggccttgacctctccctgccagaccctgctaacgatgccagcccctcctccccaaacatcttttgcattttcctacctctaggcctttcttcaggttatgctcctctgcctgcaggggatgcctttccctcagcgtcagtcctgccagtggcctctggtaaggagtagcaggagatgacaggggtgagcagggctcatcttggctccctggtggaggcgatccttcagtcacattcctaactgggggtgcttgaaagggtcacactgagagaggctggggtgtaggctgagctgcagtctgacaaatctaccaacatagagctacctaggacacagtggcctccatatggaaactgacaaagcccttggttccagctagaataatcagaggagctgagatagcaccaaaaggaatctcaggaacagaggggctgaggtggctcagatggtgggggagaaagttcctagggccaagaaccctttcaaagtgccaggcattgaatggccttgtgggctgggaggagcttcttccgggcctgctctgcttataattttggcctcactgactaccccttggatccctacagcagctttctcactgctcttcctgccctgtactgcaatccactctccacattgtagccagagttctctttctaaacacaatctgatattccacagaactcccaccttgaagccttcagtgacgttccatctccctcaagatagagtccaagcacctttggccaggcttgcaggcctctctgtctgacccccactcacttctgagcctcatcccttactctctgccttgaacttcatgctccagtgatcatctctatgctgcctctatgccttggcttatcgcttcccttcacctggaatgtcatgcccacttctctcctaagctattcaagattccacagaggtatcaccttctcttctttaaatctttgtgtgtctctaaaagttggaatacgggctctgtctgaactcatgcagcagtaaacctgcctaatctgtgtacttgtcacaccatagggaaacttttttttttttttttaaatggagtctcactctgttgctcaggctggagtgcagtggctggatcttggctcactgcaacctccacctcctctgttcaagccattctcctgcctcagcctcccaagtacctgggattacaggcgtgcaccatcagcaccggctaatttttgtatttttagtagagacagggtttcaccatgttggccaggctggtctcgaactcctgacctcaaatgatctgcccgcctcggcctcccaaagtgctgggaccacaggcgtgagccaccacgcccagcctccatagggaaattatatatgtggctatagccctcttggggttggaggttccaagcccagtgactatatttgtcacattgtggatgatcatcagctgtttgttgaataactgaggggtcactgagcccattaaggatcaaatttcaagtctctgtgtcttgggactcctgaggccctctgcacagaccctggaaaggaatgggctatccctttgcaaatgctgccaccttgtggccacagccagaagtgagtgtagccagaacatcacctatcaatgtgagagctggaccagacaagtattacagtaaggagtggggaggtagtaggtcccaagggagtgctcagaggccagcacctgtgtcctttaaacattcttgatgagttccaagcagacagaatgacaagaacaaagactcagaggcagaaaatcttatggcacttgggaagtgtgactgtggtcagagcctagggtccaacaggacaaggccaaagacggactaagccatgggcaggggccaagcaaaggggtcctttaaaaccacccagtgggctgggcacggtggctcaggcctgtaatcctagcactttgggaggctgaggcgggcagatcacctgacgtcaggagttcgagaccagcctgaccaacatggagaaaccctgtctctactaaaaatacaaaattagccgggcgtggtggcacatgcctgtaatcccagctactcagaggctgaggcaggagaatcgcttgaacctggaaggcagaggttgcagtgagctgagattgcaccatcgcactccagcctgggtgacaagcgcaaaactccatctcaaaaaaaaaaaaagaaagaaagaaagaaaaaggaaaagaaacaaaaaccaaacatctagtgaatgtagtgaatgaacttggatcctgtcctctaatcttggcatggaggctaagggaatagaagaatgacaaggtcagatctgagtatggccccatattgggggcgggtggctcaatattcattcatttactcactaaatatttattgagtacctactatgtgccaagtgatgttcaagatccttaggttacataagggaatgaaacaaagattctttcccttttggagcttacattctcatcaggaggagacaataaacaaaagtattattttaaagacgtaaatatcatagaggttaagaaaaactggagtgggggaaaggcagggtaaggaagatcgaaagtgctggactgcaattttaaatgaagtggccagagtaggcctcatgaaggtgacattttagcaaagacttaaaggagggagggagataaccatgcaagtatggggggaagagcattctaaacaaagggaatagccagtggcttggtttgggggagagctccaaggggagtgaagcacagcgatgagtatggaaactgctgagcgataatgagtggcacaaaccaacagaggctttggggatggagacctagaaggcaaaatgggcagaacttggtgcctgcttggatatgggaggcacataactcctccaggcatattgtgttctgatgtccccatttcaagggaacaaacacaaggtggctagacatgccacctctaagacaggtagtgagaatgctcaacgcattccatcctggatgactccattgccgaccagaatgctccccacctcacctttcccagcctcaactcaccagctcatcttccgtgtctctgtctccagaccctccagccatccactctcatgcccacaaatcctatcagctccaaaaaccctcaaccctattaataccttcccatcctttagctgtggttatgggttaaattgtgccctccactcccattgccccccaaccaaaaccatttatagatatcttcatccccagtacctcagaatgtgactttatttagaaatacagcctttatagagatagtcaaattaaaatgagttcatagggtgggcccttatccaatataagtggtgtccttacaaaaagaagaaatttggatacaaagacacacaaaaagggaagatggggtggagacacagggaaaatgccacgtgaagagtggcatgatgcacctataagtcaagcatgccaaaggatgctggcaaatcaccaggacgaggcaagaaaggattcccccacagatttcagagggagtgtggccctgccaacaccttgatttcagacttccagctccagaatggtgagacaataaattctgttgttctaagccccccagtttgtggcactttgttacggctgcctaggaaaccatatagctacaacgttcctgtcctaggatagtccatctcactccaccccatcttcatggggatctccaggccactgactcctgtcccttttccctcacttccctcctggcccagtttggatgccttggtcgttcactacatgtactctcttgctagtctcctcatgcatcttccctcattcctacctgtcttaaagtcagtctggaagaatgcatctgtgctttttctgagtcctcacctatcctgtgggtgctgctaagagctcacacaccctgaccaatgaccctacaagttcatggtcaccagcctacactgggcttttagccctgcctggtcattatcctgtacctcttctagtcacctctccctagtcacctctcccaactgcaacaactatctcacacatccttccctctagcttccttgccccactctcagctatggagctggactactgctttgaggagataagaggccttctgaactccctcatgtcccatcttcacctccagacctatctgcacacatatctgtcctcaactccatccctcctgttacaggaagtcatccctcctcttccctggggaggcccttacctccatgcttctggatcccatttgcttttacctgctcaggaacctcatgctgtgagttatccacaatctcctgcatccttcatctctttccctactggcttcttcgtctgtcagcctataaacatgctcaagtctctgctagtatattaaaaactacaacaacaacaacaacaaaaacaaaatccaacactctctgtactccacattcctctcctgctatctatcacccctttctacccattcacaatccaatttcttgaaagagctgtctctacttccttaccttttgtgccaagacgtgaccttatggtggcagaagccacattgcagtgaattaaggagaaagtggacagaactagacacaactcttgtgctctcttgtaaagctggtcagcctttctgactccaaagggctcatgcctctggtcccctccctgccattcctcctgtaggccccagctgggccccgtagagaccaaagatggagagaaaatgcccctcctgggactccagagctcgcaatgccaccttcagcacagatcaccataattattatatgtgatccctccaccctggccatcagcagatgggatgagatcatcagccaaaggtcaatagtctgcaaggtggcctggcctcaaagctctggagttccaatgatactcagagaactgctgatggcagaagacacacaagtgaagatgttcagagaaaaagcggtatgcagaagctgagagggaacaaaattcctgaattagctgcaaggagaatgggggaagtcagtcagttgctggcagcaagacacctgagtcactgccactgcagccataattgcctttaggagtgttttagatgcttcacgaccctgtgcatttgagagacctgctcggcagccactgaggttttctctcctaatctcctctgaatccccatcacctacccctcccccagggtcctccacatcactcagagctaacctatctttgaaagcaaaggatgcagtatttacagttttgaaaactgcctgctggccctgtggtcacctcacccctcctctaagcccttatcaagggcttcagtcctccaattccctctccttctcaatccctcctgcctggtctcagttaagtatcactgcaacatccttacattgctgggcccattctactacaatgtgagcccagaagaaccccaaccttctgctttcctcatgactggacacccaggctgctggacacattggagaaaatctcaagatccatgggagatccaactgattcaattcttcagagttcactactgcccataatcctgctctagttcccgcagcaccccctgctactgatgcaaccttcatttctcttctcatgcccccgaccctgagagtttaaggccaaggcaaagcagaaaaggagcagcaaagggaaaaaaggcaaggatgtggccgggcgcggtggctcacgcttgtaatcccagcactttgggaggccaaagcgggtggatcacctgaggtcgggagttcgagaccagcctggcctacatggagaaaccccgtctctactgaaaatgcaaaattagccgggcatggtggcgcatgcctgtagtcccagctactccggaggctgaggcaggagaatcgcttgaacccgggaggcggaggttgcggtgagctgagattatgccattgcactccagcctgggcaacaagagcaaaactccatctccaaaaaatatataaataaaaataaataaataaataaaaggcaaggatgagacctggagagagatgtgggattgaggttttttttttttgagatggagtcttgctctgtcgcccaggctggagtgcagtggcgcaatctcggctcactgcaagctctgcctcctgggttcacgccattctcctgcctcagcctctcgagtagctgggactacaggcgcccgccaccacacccggctaattttttgtaattttagtagagacagggtttcaccgtgttagccaggatggccttgatctcttgacctcctgatccgcctgcctccacctcccaaagtgctaggattacaggcatgagccaccatgcctggcttttttttttttttttctgagatggagtctcgctctattgcccaggctgaagtgcagtggcggttcactgcaacctccgcctcctgagttcaagcaattctcctgcctcagtctcccacgtagctgggattacaggtgcccaccaccacgcctggttttttttttttttgtatttttagtagagacagggtttcaccatattggttggggtggtctcaaactcctgaccttgtgttccgcccgctacggcctcccaaagtgctggaattacaggcgtgagccaccgcccctggcctttttttttttttttttttaagatggagcctcattctattgcccaggctggagtacaatggcgtgatcttggctcactgcaacctctgcctcctgggttcaagcgattctcctgcctcagcctcccaagtagctgggattacaggcgcgcaccaccatgcccagctaattttttaatttttactagagagggggtttcaccatgttgaccaggctggtctcgaactcctgacctcgtgatctgccctcctcggcctcccaaagtgctgggattacaggcttgagccaccgcgcctggcctgttttgtctttttgagacagagtcttgctctgtcacccaggctggagtgcagtggtgcaatcccagctcactgcaacctcttcctcctgggttcaagctattctcctgcctcagcctcccaagtaactgggattccaggcatgcaccaccacgcccagctaatttcttgtatttttagtagagatggggcttcaccaagttgaccaggctggtcttgacctcaagtgatccactcgtcttggcctcccaaagtgctgggactacaggtgtgagccactgcgcccagcctgttttctgtttttgagacagggtctcactctgtcatccaggccgaagtacagtagcacaatcatggctcactgcagcctcaacctcccgggctcaagcgatcctcttgcctcagcctcccaagtagctgggactaaaggtgcacaccactatgcctagctaatttttagatgtttttgtagagacagggtctccctactatgttgcccaggctggtctccaactcctgggatcaagcaatcctcctgcctcagcctcccaaagtgctgggattacaggcatcagccactgtgcccaacccaaagatatgattctaagacaggaaaaagcatgtttgcaaactagtggagttgaggataaatgtgtatacagacaggtctggaggtgaagatggaacatgagggagttcgaaaggcctctgtctcttcaaagcagagtccagctccatagctgagattggggcaaggaagctggagggaaggaggtgtgagacagttgttgcatttgggataggtaagtcattaataacgataatagcaattaacacttaatagcactatgtgccagtgtggtatctaaatgtcatatatgtatatttccttatgtaatctcataacaatcctatgtgataaacactatcgtattcctgttttatggataagaccactaaggcacagagcagtaacttgctaataagtggcagaccaggattgaaacccaggcaggttggtaccagagtccatgctcacaaccactatactatactgtatgctgcagatggagagaaatgtctgacctggcagtgttcctaaaccaggccaaagcaggggcaaataactatccagggaattagatatattcagtactgactgaggcttcttgtagacactatattctcccagcaccggcctgagcctactactcttcctctgtcttcacccgatcagccccccggggtaagggcggggactcccatttctttaatcccactccacatagcaggctgagtagaggctcatcaaataggtagaaactttatttcctaaatccctccctggacctctttcagaaggcagttcaaatgcactgtaggtagaaggcagaggaagcccttatttagcaatgcagaacttggcagaggccccacatctgtcattcttcacagcagtcccttcccacatgctagagggaaggggaagcatgatagggaggtccacttttgtggactcaaaccttgatggggatgttgagcagtcacaacgcttctcagaaaaggcacaagcaccccagacattcaggcccggagaacaggctggctcagcaggtcttcacgatcgggtgtctcgagcccttcttcgggaacgagggccacagctggagctgggcatggaacccaggccagggggtgctcctgggggttggtaaccacactcattggggtccagggggtcccggctgagcagcacccacactgcaggcacatggcggcgcactgtgagggggtctagccctgtgtcaggtgaggctgggacccagctgtcctcagtctgaaggaaacgtagcttggtgagctggtgcaggcctgggacccgccgcttgacaatctcagcgcagctgacagcctttcctgcagccctgccagaacctgagaacactacatgccgagcactgccgccctccaaccgacccagagccaaccccagcaggttgcgaattttgctgccatctcggacccgcatctcaagggtatcaggaggtagctggggcattggggaaggcgctgggagctctacagagccagctttccggtagtgctccatcctgcttgctgtcttgtctgtgactctcgctgcccactgcagggaagcatcaggtaagtgctaagcaatgtgccccaagtcactgtccaccatcccctatctccatccaggatatggccctgctccccgttccagacttcccagaaacaggatcatgtccccttgtccttagcatctcgtgacaaggtccatgcctgccgcagtctgggaaagggctgtaccccatacaccccgttcctctacaagggccataacacccccacctccaatcctggagtccaagaggcaggatcacattccccaaacactgagctctttgggaaagtgccaggccccctcatcctaaactctccaggactggggcttagccccccttatcctgggctccttggaacagggcctggaaccctcaccccagactaataccaatagagagcgctcctcacttggggctctacaaggacagggacaaggcccttcgccccaggccccccgggaaacagccaagaacccttatccagtgctgcccccaaacagaacggatcccctctcccagagattaccaaggtcaatctatatcaacccaggtcactcagaaactcgggggcagaggctcgactcggagggctgctcaggacattccaagacagccttggccgtttccatagccacggtctcttccagggtcttgccggccgctgcagccaagccacaggcgtcccccaaccctacgagcgcccctctgcccgcccggttccggctctcaccgaagccgcctcacgcacgatccggagccaagatggcgcgcgggcccggagcgcactaaggccgcggggccgcgcacgccccgagggggcggggcgtgaggggcggggcgtgagggacgggccccgaggcatctccgccgcggaaacaacccgctggccagagcgccccggaccagtcccagcggggatgggaacacgagcgcagcctccgctgttacgccccggagggaggcaggacagttccctgcggtagaaaggagcggccgtcggggaagtcctgcgtctgaaggagggggatctggcccttagaagatgggagggctggcgttagctgttgagcaaacggggagctgtgggtttcaccgtgggtgcgaacaaaggcttcagtttgtagtggctgctgttgcatgggcgagagccttagttaccgacttctgagcttcgacctcatcctgtgggcgctggcggaccacgggggttttccgggttttccaggggccagtcgaaccagatcttgtgcagggagccgaggagggagtggtggggaaatcgggccacagcccctgacgacacctgccgcggagtctgggggctgagggggcgggggtgcccacggagcctagtatggtgcctggcgtatgaccggcgctcaatatttgttaaatgatttgttacattaatgaaccagagaaatcgcggtgggaatggaaaaaagattaagttgggagatactagtaggttcagtgacgagttggccactgtttggacggagaagtaggaaattccgatctagggtgactcccaggtttcagatcggtttggacacgctgaatttgaggtgccggagaggtcgcaaaggcggggcggccaatgggccgttggaaaaagggtctggagccagagcactccctgagaccacaagggggcagcaaacatttggtggactacggtgccaatctgtttctcaacatgcttgccccagcgcccctacttcccaacttcccctccccaggaccaaaacttcctgcatccaaacctcttaggctgcccttctcggctcgtggcagcaggtaggctcccaagagagaaatttaaaggcctttgcacatgctgctcccttggctctgaaagtccctggatatcacttaccaggtcccttcagtcccttttttcttctgtgtccacctgataggactgcccttttcctttactgtctatcctgctagcccgtgcagggcagggattagccctatctactcccatcatctgacatttggaggtctcttattggtaaatgtgggataaatggatacataaaggaatatcctattcacataatcaaatgagagccttcattttgagagctctacacctgccagaaagccagaccaggatgagtttagttcccagtcaaacgattaggcaggctgacacctctgccccctaccacctccatcctagaccaagaagccagcacactcctccagtcatctctatggtttattgtcactgaaataagaacactgattggggggagggtgggtgttgcaaattgcaaacctcaggtaacctgacctccaactttagagcccagagtacagagaggtgggccttgaagccaataaatacaaagcttcctctgccttgtaacaaaggcaggttggcatagggccctggagcctgactgcccaggcccactttgggatggggagcagctatcactcctctgctggcttcacttgcgtggcggcctctagctggcaaagtagctcatcccactgcacgaattgcttggagagaagcattgtctggttgtaggtcaaggtgagaataggaatctgagtgaggaagttcatggaaagtggggaacaggcaaacataggtgggtaggataggatgagagagagctagaggtggaacaaaaggacagggcttagggacagcacccattagggtcctagttgagcagaaaggatacagtcttgttgtattcctccaggagagccttggactcctcagtgatttccacacactggtcctgtagggccaaagtgtagcacagaaaggaactgggcaaagcccacatcttccactaaacttcctgcctcatcacggactcccactccccatggagcctaaaaaagatgacgcttaaccccaaagccaaaggagcaaatggctatgggagcactttcaggctccaggatgggtgccctaacagcagtccaaagacggatggaaaagggagtggccatacattaagtaaacattccccctagtgtatgtctcttcttctgtccttatttctgaactctctttgaactttacactagggcctgggaatagtgaaaaaagagcccctctacccacagtcaatgacataccatccccaggaattcttgggtacctcccagcctcaaccttatggtgtcccaataaagtcctgctctggaagcagaaagtttgtacatgaacctaggcaagtccctttacctccctggggcatatgcaagataaaggataaaccccaaagtttctgctgaggttacaacagaaaggcctgaagctgcttccccaggtggcatgagttgggatgaggtgctgcgccacctccatcttcgcttctagtcatctcccctcccatacctgctgctgaatgtggatctgggccaagcgctgcaggcgggcagcatgctcaggaacggctgtaaaacacaacacacactgctggatgatgcttgtgccctcccccgggactgctcaatagtctattaggtctttggcaggattagagtattgagtggagaagctaaacaaacgccagacttcatcgactgctgcagctggaggtggcagtggggctccctgcccactccagtcttactccaggacccataggggaggggaagatacatgtcccgaggagaagacaaaaccttggggttcccagtgttgctccctacccccaccccagtgctataagcaggaccaggtaaagagagctttggctattataggacctgtccctttgctctcagcccagcgggggcaggtctgcctggcttagggaacagggagtctgaagatggatgccaggtccctaagctggaatccaacacgccaaggcttggccaccatgctgggctcaaagcttaaggctgtcagatactcagcgggcagctgatcaggggtagggagggggagctgggaaaaatacctccacagacagacaagatggatatctggattcaattaaagccactatctgcttgtcaaagtccaggcccagggactgctctcagcagtgcagcagaaagaattcacatacattcatggacctggcagagactctgatagtaaaatctgggttctgcagcaccttgaagaagagatgcttgggggtaggtagaaggcagaaggaatccccagagctggggactgctagagcttaggctagtgttctcaggcccaaattgcctgaggtcctaaaactgggctctctatcctggtaattttaattggtggatgtggcaagtacctcagggtgaggataaagataaggaacaggccaggcacagtagctcacgcctgtaatcccagcactctgagaggccgaggcaggtggatcacatggggccaggagtttgagaccagcctggacaacatggtgaaaccccgtctctactaaaaatagccaggtgtggtggcacacacctgtaatcctagctacttgggaggcaaggtgggaagtttgtttgaaccaggaggcagcggttgcagtgagtcaagatcatgccactgcactccagcctgggtgacagagtacgactctgtccaaaaaaaaaaaaaaaagataaggaacagacccaacccgaatccacacaacctgaaacacaggaaagtcttgcccatccatccacctacctccccaaccagagtagtgaagctataaccccagagagatacctttgatgtgagcactgtccagcatgggcaccaaggcattcacctgctccaggagtgcaacctgggaaaggataaactgctcctctaaagcaaaaatggacaagatagttgcagtgaagcaaacctgggcattgctaatcagcccatgtaagggcctgaggacctggcaagggagatggctaggtcctagagctttggacagtgactctgtccactgcccccttctctaggtgcacatttccatcctgcccccaactctcctggatgcctcaggtctgacccatctgagcctaatgagcctctcaatttctccaaatcccacctttctccaaggtttaacttcaagacctcttcttctagcctcctatcaacaaactcagggagcacctcctaggccacaaaatacaaaatacttcagacttggatccccacacccaagggtccccaggaaggtctaaaagagatatgtggcagactacgccaggcaggttctgctcagtggctctcagaggcatagacaattatatgctggaggagagctgggacagtaatggaagagtccagtgaggagagcaggatctgggctaggccctgtatgcacacgggatttcaatgcactgaaatccatagagtgggaagggtattcaagagagaggactgttacgtgcaaatgcttagaggcaggaatgcctgtcaattagtagaggagaaagaattcaattagctgaactggagtagggagagcacaggaaacagagttggatgggagggtagacccggccgactgaaaggctttgaatggcagactatggaattaggactttatcctagcaaggcaggatacactgaggttccaaagtaggcaaagtgacatgttgggaacatgtttttgtgagggtgagactgaaccacatgcaggaagggaaagattagggtcaagagtatgatttaggagactgtgcaaccattcatatgcaagtggatgcacaattcctgaggcagtgccacagggagggatggagaggctggactcaagagtcagtgcaggagaatatctaaacttggtgcttgagcgatggcaattgaccaagagccctttgggaactctgacagcccttttagcaggagtggcccatccttacccagtcctcagtgctgcattgcactgatcagtctcctgggagactgatctcccatctgggttataagctcaggtccctgaagcagggaccctgtctcccctagctctggctgtgcccatgagggcagggactttctggttcaccactgtatgcttcctcacttaggaaagaacctggatcagagtagatgatcattatacatcttttggatgaataacgaagctcaaaaaaccctagcttatagaatggctgactgattagcagacattgccaaggcaatagttcctgcctagaactctgacactgcagagctttctcacatgaaagaaagatgttgaaactcatgccaatactctctctgttctttaatggcaatgctctctgcccctaccctttcctagaagctcctctgtctagtgagcagaagtgaggaaggttctgaaggaaaatccaagaggcaaactattgatagaacactggactagctcccactgtggctggcaccatggcctgcagctggtgcagaggtatactgtactcaatgtctactagaggatccataggatcctcaacccagaaccaggccacaattccagagagcagcatccagggcttgtatccaccagaagactggatggagaggccatctctgtgcttaacctccccgacgacccacatcctcagatgcatgtacacatgtagtccagcattacctgctaggatgaattgcagcttagaggcatcaggtatggcaatgcggtcgatgtactcaggatccaggtacttgatcagatcttcaactagaaaagtagcaagatggaaaatggaatagggttgggcagtagagctctgccttatgaaggatgtttgacaagtggtcagccttgaggagagccttcaatgaggagctgatacccaacagtgccacatgagcagggctccaccaggctccctatccccacttccctatctctccactctcttctcttgaagtctcttctgacatcaagcttcagagctgaaaacacaattgccaccaccactctgggcccttagcattctcactgacctatcctcaccaagggtagaaggcctcttttctcctagatgtcaccgagacaagccagagctttgtctaatttctagttactaagcaggttactagttctgacataaccaggatctcagtgtctaatttgtttcctgccccttaaccttgcctgggaaatcccataccctttttaccctccttcaccaagagaagctttcaggtagagctcttatttcataacttaggacactgtcatgggcttgaggtttagccacaaggtagaaaagcacaccacaatttaccaattgaattgggtgtaggtaaatgtcctcataatctttctatccccaaagggctattccagtgacctaatgaactgaagggctagggccaggtaccaggaagctgaggggtgggatgtcgggcaggcaggcacatcatggaagcacttactctttttgtagagaatcttcaccctctccctcttgctggaaatgttccccaaagccacctgcaccttgaccaggccgtcagccacctgtaagggaagtggtggttaattccaagatatactacctggctcaaggcttggaaaagttaaagaaccctcattctcccagactctcaaacctgaatcccagtaatctaggttggcctcccttctctaatattaaaggagatgcagccccaccttccaggaatccttaaagtggagagagagaatcatgtactccttggggagtttcaagtctgatggggaagataacgaactattttagagtctatggaatatagagtatacacaatatattttctggttcctaagtcctgggtttgggcatgagaaacacaagcatcctcattcaagccttcatgacctctagattggctggcctacctctagccctatcccctccacatcaaatttggtgtgggagatggaagagtccaaacaactgggatcccaatgatggtgcaatcactgaattagggaatacagaagagaatcaggttaggggagagaaagagtacagttaagggcatacagattgagtttgaaatgactattggatatccaagtggaaaagtccagtagacaactagacataaaagtctggttctcagaacagaaatctggaccagagatatacatttgagacatattgacatgtaaatagtaattgacaccatggaagcgagtgagcctacctaaagtttaggggtagaatgtaaagaatcccaatattaaagggtcaaatagaggaggagactaaggaagtgactgcactcacccagtttttagggtagaggatgaggaatgccaggggggaggcaaataacactgagaagcagccgtaagaggtagaaggaaaaccaggagggtcagcgtcatagatcccaaaggaagagaatgtttgagaagttagttctgaaatgggagcacagtctgctagtgacaactaattcctcatttagtgtacaaacagacatcattaccttggacgcacttagctccaggacatgcctgacacgccatcagaagttcttcaataaaagatcttcccatagtagaaaggggaggagagggcacctgagcttcgtagttgtcaaaattctgtcactggtataaagcagtgctagtccacatctgaatacccagtacccagtctcttcccttcctaatcctggtcctttcagacctcacttctcaccctttggcgttttccttatccacctctgctctgcctccccaggggtcccatggacctcttcctgcacaattcatttagcaattaattatgcagggctctatgacgggccttcctatcgtggacttcagctcttattgaaactctcattagtcagcttctcgtgtcaggtctccccaactatagctcgcgagggcaagggcggagtctcagctacagtaggtacggagcccactctcaggctggcttaaccacccgcagagccccctatgacagtggaccccgatggtccaatcagctctgggaggtcccggacctttgttatttcaaaggccgggctgcggagaacaggactggagcggttgcatcccgagggccaggacagtggaccgctcttgctctgctccagaaagggactaccggaccagactactcaccttccgtgagccgcgcgccccgcccggcccgtacacccagcgctccagctcttccactcgggcctgtagccgctgcaagtcagtcagacccgccatcgctactaccgacccacctcggccaggaaacagccagagggcgggacgtcagtccaaataagaaggctagccaatccgagtgcacgagcggccctttatgcaggaagtgacgccatgattcagcccttcagtgccggaagttactcctcggagctgtcaaaagctgattaccttggcaacggctctgtcccatccctcccctggcttccagtgcctggttgtcaagataacctttgcttttcgggtaccaaagagctttccccacgccattccctgagtttgcacgtgttccaggataggtccattcccatagatacatcattaggaggctccgtgagttaccataaacttttggagtcttctaaggttcagttgattaagtaacttgcccaaggccacccagcactatagggacagaaacaggacttgaactcaggctgtctgattcaagagacggagctcttctattctcacgaaactttgctattctgccttaacctctgaaagcccctggcccaaggcagcctatgttccacatcattcctccccacctcccatacccaaacaagaaaggaggcatcagagagggagggaccgcagctttattgtctagatgtgacctgagggatgtctctttcacagtaccccaacccaacctttcacttggctcctgaagagaagcagggctgactcaaaagggtagggtcctagagatgacattgacagagggaacacatgaggagatgatggggttactggaggaagaaccctttggtaggtgctgctggaattggccagggaggaggtgccctccctttacaatgaggtaggtgtaacatcaaaagaaactttggttttaagtccacatggcatgggaggtgtcgtcccctcccagggctgggtcccagagtgggcagccagaaagaatcaaagcagggggtctcctcccccctcagcaatggggctgggactcttagtttcaagtaggacctctcagggcaagatgctggaagggggccctgtggagggtgggggtgcagggttggttggaccctgggaagctggcacaggctggcggcgggcaaagaggacacctggcaaaggggtgaggagatggtagagggcaaaaacaagcaggagggggtagggtatggaaacagaagaggggaacaaaagtaagtgttctggttgctagaacactacacacgtacccctgccacaaaccaatgaggacaagctccttgggtcagtaagagactccattgctaatgtagatacctcaaacaggtaaacaaggaatatttatgctgatacataggcccaagacaaggcagacatcccctggtactcatgccagagaggtcatgccttggatcccatgccatgtagacaccccctttaacccatgccagtctagcaatcccctgaactccatgttactcatgcaagtttagatatccctgaacttcatgctagtagaggaagtctaaaatccccatgccagcctaaatacccctgaccccatgccagtgtagacagcctgcagtacccataccagtgtagaccaccccgttgatctctagggccatgttgatactgctgatgccactgcctgccagattaagaggcccatccagccgctcttctgtccaggagggggcagaggaatcacattttggagaatcagacttctgacttattaatagaatttcccccctccatccccgtagacagcccccattccacttcagccccacacctatactcacacagttgtctacagaggaatctgtctgccccgtctctaacacctatcaggtgatgggtcaatcctccctcagtgtacagtcccgaagccccctcacaacaagcccctcctcacccctcaggggaaccttgccacggggcaggaaactggggggcatgcaaggtcgaggtgggtccataggccccatcagcacagctctcttctctgggggctcctctggtgccaatttctcccgtgtaggtcccagtgccaggcccacaggcagtgccagacaagggtttggaattccactctcctctagaagaacaggttattcagctcccctggccaaaccaacccatctctgcattggttctcccccaggcctgggaccaaaagaggtaactcaagctcatgtaccaatctctcatccttcattcgatctatcttcccatccagagcctcagcctggaagcgctgaagcttttctctagagcccaaaggatcaaggtacatgtctagacaatttgggggattgtttttggctgttggttattaaataatccaggtcacaagaggcacaaactatatgcaaataggagcactgggtggacatgtgtggaatggagttgctgtgattaaaggaccttttcctggccgggcgcggtgactcagcctgtaatcccaacactttgggaagcggaggagggcggatcacgtggtcaggagatcgagaccatcctggctaacacggtgaaaccccgtctctactaaaaatacaaaaattagccgggcatggtggcgggcccctgtagtcccagctactcgggaggctgaggcaggagaatcgcttgaacccgggaggcggaggttgccgtgagctgagatcgcgccactgcactccagcctgggcgacggaacgagactccgtctcacaaaaaaaaaaaaaaaagaaaaaaagaaaaaaaaggaccttttcctgaacttcagcccctacacttcagtaacccgtaccccagagctctcatttggccgcaaacctcaggacccctaaaccttagtgacccccaaacctcagaagcccgtcagaccttactgctcccatacctcaatgcctccccagacctcaatcctcacattttaatggttgctatatcttagcgcccacctaaacctcagagaccccgaaacctctaccattctcccctcgcacctttcttaatagggcttggactcagctgagcgcatgcatgcgctggcaggccggaggtggaaccctgggctgggccagggctggactgggtcgccggaggggcggggccgggacccaaggctgggtcctgagcgccccgagggggcggccctgccaagccgaagagcggagtagcggtgtaagcctggcgacggccctcgcgccgattgctgtctatggcggcctggagctcccctggggagctgagcgctcgagtctcgcactcgtacgggtacaggtacttcatgtacctaggggcggacagcgggcggtgagtgtatgggaggctgcacccagctggtcaagtcccacagccggacgcccgcccggggaccctcccccctcccgccccaggccgaacccgtgcccccttcccttccgctcccgcccctggcctcactgggtgcgtagagtgaaggcggccgaggtgatggtggtgggtaggctgaggccgcgcgtgacttcccgccacactttgcggttgatgacttccaccaggccgcccttggcggtcaccaggcgaaacagagcgtacaggtcgagcacctgcttcgccatgatgggcacgcggttcactggcgtccctggtggggagcgggctgccgtcaggacactgagacgaagaccctgtctgcagcatccccagggactgatacaggacgggagccccggggagggcgggaaagagggagacttgggcggagcccacagaaccccagggtctctgttttagcaagaaggacaggaggctagaacgggggggggcaaagttctgtaaaaggccagatggctaaatggtctctgtcacaactactcaacgctgtcattgtagcctaaaggcggccatagagaatacctgaaggaatggatgcggctgtgttccaataatatagactctgaaattcgaatttcaccagtctggagatgttattcatctttaaattttgtcccccaactatttaaacaagtaaaaaccattcttatcttgcaggcagctgggtttggcctgcaaactttagtttgctgtcccctggggtagaccgcaggaaggactttcggacagtgctgaagagccggcctcccctaagcagcagcccccgggtggctgcccaaggctgggggatgacgggcgggaaaagaatcaactcccgcctcggcccgccgactccgcagcccgcgccgcccccgccggccagtcctgacaaaggggcctgtctgcgctgagcgatgggccccagttaattccttaccgatcccgtccccttccgccgcgcctttggacccgaacaattagctgcggcctgattaatgggggaaaattatcggccccgcgggaccccctccccctgcagctggagcggtgggaggcagggaccgaagggaggcaggcaatgctgggatcggttcacacccgctaccccagtggaggggaggcctcgaggaagggccagagagggaacactggaaccagaatgggcctccctgggaaccaaaaggacttaggtccctactgagggtccctgggaaggtaagggaccagaaagaggtgctggggtgagggccgggagtgggaagtgggccagcagctgcactaaaggcggtgggggggcggggggtaacagggcagccagttaattagcggcttatcaggctgcgctctgagtgagttaattagctttgcagagagagataatgagtcagtgttcggtcccatgaatctgggaaaaagctgaagtgatggactgccgcccaccctgatgccctcctggacccttcttgcgttctcctccagctctgacattcctggaaaggaatttctcctcctccacagcctcaactcacatagtccccagcccaccccttgggaagtccttccttcagtctattcctagtccttcctgctgtagagcacgtgaggtgtcccctgcctgaatcaagggaaaacagggaagacatggctggaggaaggaccctggacacagaaccaaggcattctcagccctctgattctgggaccctcccaccccaggccaggctcccatcagttccccctcccaccctcccaattgatcccaaactcattaacctgccagtgggctggccactaattaatcctggggtggaggagggggacagggtcagaggttaaagacgctatcgagggcccaaaggacaggtgctgccaagctcaggcctcagcagggagaacccaaccgggtttcctcaaacctggtaagggggcaattccagggcccttcccccacatcatgccactcacccctcttttgcatgaagctaaacaggtcatccagaaattccttcctcttggggtctgcatcgagctcatacagctggggaaggattggggtcattctacagctctgctgactccccctccctcccttgaccccaattcaggttgtaccctgaacaagggtccctagctgaaggagtcagtaggatctcaatgtggactccctgggattagcattaggctctctccgcctaggggcaggcaggggtgcctttttctagtacatacagagactggtactggtagtggtaccctgggccattcaaaccacccatctggggagtaagggaagagaggaacaggatggagcttggaggctcagcctgccctctggggctcagtcttcccccaccctcatgggaaatttagagccccaggaaggaagctaggctggggttgtataccagactgaggagacaaacatatatctgatgctctgattcattcatgcaaggacttcatggtgttcacatccctgtatgccagggctgggggtgactgaagggcagagatgctcctaacccacccctcaggcagggtgatgctcagaagctcctgggttgacacccagggcccttcagctcatcttctggtctgatttcctttcagaggcagttaacctgcctacagacgctgctatagaggcactggccctgtaccacctaggacctgtggggtcagagtttaaaggatccacaggccatctcaactcttcctggtaatctggagtcccacctcctgcatcaactcccgaatactagtcaagagaaccaccccttgagtcactgacccagagtaacagtccttgagtaacacctatagataatattccatcactccatccccataagaatcctcagagtataaaacaaacaaaatatccatgagtaataaaccccagatccacctttagagccacattcccacaaaaattgagccctaagtctaagatgttattacttagatccaagtaataacccctagaattatacctaggagaaacagtctctagaccacatatccagaacagagccctgagtaacactcagttttatactaatgccagagtactagtcacagatcaacacttttggaatatacccagaccaacaccctacaagtaccagaatagcacacctggagacacagccctgtgtaacaccttcatagtgacagccttgagtagtgtccttagaataacacccagaagggttctccagggagaaagaacctcagattaaggccctgtgaatgaagtcttgtcattctgagttgcggaggagccctttagtcatactgcggctgtagtggccatcccaggctgggaactatcatccgtggaactcaggccagggagatttgcatggggccagggttagggaggaggtgacaggaaagaaaggaaaggtgtatcagcctgccaagtgagtgataacctccattctgcttccaaagacagaggaagcaactgggtcacccccactggtcactggaatatgaccttgtgacttgatgtgtgagaaggtaggcagggtggtgctggcattggaatccccagcccctagactgccttccctacacctggctgggcatggtggctcatccctgtaatctcagcactttgggaagctgaggtgggaggattacctgaagtcaagagttcgagaccggcctgggcaatacaagaaaaccttgtctttacaaaagaaattttaaaaaattagccaggcatctgtctttccctccctgtcactatcctccaatcttctctctgtctttctataggtctccgtgtctctgtgtctcttttcatcttgcagagggtggtgggggtgggtgggctgctaatcacctggctgtgcttttgccagaagagagagatagggaagagggccggacattgagggcataggtcctacctgcttgaattgttcctcgtaggtccactcgtggggatggagtccagggggctggctagaaggcgagctggggccctgggcccctggacggctctcctcagctgcctcctcctctgccccggcttcctcccgcttctcctcatcttcttcagcatcttcctcttcctcagccccaacattccccaaggccccctcaggggcctgtagggtccggtggtcaggcaggggaggctgcggtggcagcagcgggcatgcaggggccaatggccccaccccctgggccagccgagctgcctgctgcttctgcagggcctccatgacagcttccaggcgcagtcccccagctgagggggtccctggtaccaggcgggaccccgaggaaagggccgcctgtgggggagggaattatgggtgctgggccctactgcccccagagccccccaacacagggggaggtggtcatggattcagggaagtgcaggggtcacaaggaaagaggcagaaagatggctttcccagaggtgaacagaaacaggaccagacccggggaatcaaatggccattgactgaggactggaagagtgagcagatcagagagggtgacggggaccaagagacatcgggaaatggtgaaggaagggagagagaaagaaaaggagaagggggatgtccgacagagacaatgacagggagatgtgaaaatagagatggaacagggtgacaaggagagacagaagtggggtgacacagagactgagagggaccaagatggagagaggcagaggtatcaggcagagacagggtagacagacagagatggagagagatagaatattgggtagagagacagggagggtaagagaggtagaggagcaacagataaaagacctaagtgacaggtagctcagagacggacagagacggagagacagaggcggagatagacaaatagacaggggttgagatatagggaaagaccaagacagcaggaaacggacagaagagagagattagaaagagacagttaaatagagagggaccgacagagacagggacgggaaaaacagccggagagaggggcagaggacagggaccaaggagtcgcggggatggagggcggtggggcagagcctgggacgggcacgggaccggacgggcaggaggcggcagagtcgcgacgcccggctccctcgctccgggcgccccgcggccgcactcacctgcctggcgtggcccgctgcccctggcgtctccacggcgctgcctttgctccgcccgcctcccaggcggccctggcctcggctgggtcccggcggtgcgctcccgccgccctgtgctcctctggcggctgtgcgctcactcgccgcgctgcttgcggcggccgcgggggcgggacccgagcggggcagggcgggcggggcgcatggggagaggcccctgtgggatctgccgctctcagctccccaacaggtgtctcccaccttctttccagccagcttcgggggcagggggcgctggaaagctgcgggggaatcgtgcgaaaatgagggctctaccgccaccggactccggaccagagccccgcccagcctcacaagtcgtgcccccgacacccacactcggcactgggcttccacatacaaatagccgcatccacacaccaacacaccttgtcacaagttcaggcgcatagcttcccagtctcacacgttccgtgacatggtcaccacagtgccaatggcctgggcacgctgtgatcaagaggggaatccctctgcaagatgctggggtgtgcgtggagggtctggtcagaatagaagcttctccctttgccttgagcacccctgccctgcctacgtgctgtcccccactgcgggcatgctgcggtttgcacacatgagtgcacatggtggtggacccacacctatgcactcacacactctcagaggtctttaagctgactgcacactcaccgcatgcagatacacagccacacacagtttttgtctcttttgacattctattatggaaattttcttttcttttctttttctttcttttcttttttttcttttttggagacagagtctctgtcacccaggctggagtgcagtggcgcgatctgggctcactgcagcctccgcctcccaggttcaagtgattctactgcctcagcctcccgaatagctggaattagctgggattacaggcgtccaccaccagacccggctaatttttgtctttttagtagagatggggtttcaccattttggctaggctggtctcgaactcctgacctcaagtgatccacccaccctggccacccacagtgctgggattacaggcatgagctaccgtacccagcctgtattatgaaaattttgaaacatagacaagtgaatagtataatgaacctctggtactcatcataggtttgtttcaacagtgaccggcacttcgcccatgttcacacacattctccacctatgtgtgcctgactgccacatctgtatctccagaccccctcagcccactgctttgcgccctccccctaactattcccaagacgctgaaatgctgttccttatttgggggttctgagtggcaggcctctcagaatttctgctctgaacagcctggaccccactcctcccgggaatcctcatacttcctatttcttcctgtgcccaaggggaccacttaccaaacagagggacactgtgaggtgatgccaagtgtgtaagtgaacagcaggtgtctgtaggggtgacacttggcataggtatgaaactgtggcaccagagactgtcttctgacctcacacccccagaaggcaggaatccaaacacgacctttgtctcctccctcctcccttgtcctctcccaatcagtccactcctctcagcctcagcttgctctggctcaaaccaccggctcttctgaccatggtgggtctgaccatggtgggtgcctcctccctgctctccctgcctccagccgccacccttctctgcacccttctgtgcccagcttctactgcagcactaagtcctggttctctatggcccattcctggccacgctgaggctcaccccattcattcctttggctgccccagtgtcctccacctcagatttcagacctctgagcccatcagaaccccccagcctccagaagctccagtgatccaatcactgtcggtactgatgcctcctcccccatggctttgtttggtgtgagtatcctggggcagagccaagcctcctcctctgcccaaccctccctgatgctgggaggggagaatcagtgaatgtgccaggcagacagacatgccctggaagtctactggaagactggagagcagagggtagcaatccacagaacccttgtgccctgcttgctcccttttccccaccacagcttcccctggctgctcagaggaggtccatggacaaaggtaggtggccagagtcttcctagaaaggtaggaattggcttggggaaattccagttctgagtgtttggagatcgctttgtagggtttgggttgaatggtaaggggctgggcagtcactcccccagctcccaaatcaatcaagcaaagttaatccacttttggggtctgggccaggagggctacaactgggaagggctgaggggaaggaatatgcttgactgggacttggtttatctctcttcatctgtgaaaaggaaaggctgcctctcatacccgcagtctggtcttatacctcacagagacggagacagacctagaccttcagagacccatgcttcattcatccaacacatatttattatctattttttttgccaggcactgttggacacaggagcaaataagacaaagtccttattataatggtacaattctaagtgggggaaatatacagtgtgtcagatagtggtaagtaccatggagaaaaatgtagctgggttggggggaacataaaagggggcattagttacctattgctgtgtaacaaatcaccccataatttagcagcttaaaacaaaaacatttattttctcacaattctgtgggtctggatggttctgactcattgtcctctaaggtgttgtagtaaaaaggttggccagggatgcagtcatctgaaggcttgactggaactggaggatctacttccaaaatggctcacttgcatggcccttggcagggggcctgttccccccgcaatatgggcctctccataggattgcttgtatgtcctctcaacatggcagttggtttcccccagaatgatccaagagagactctttagacagcaatgctttttatggcctagtcctagaagtcacacactgttatttatgccacattatttgtgaaagtacataattaagtctagtccactcaaggggaaggaatttaggcttcacctcttgaaggacagagcatcaaaaattttgtgaaggtatttcaaatcaccatgcaagagatactattatataagtgttgaagaactcagtgagcaggaattgaaggaggggaggaagagagctatgactacgtggatacctagggacgagcactacggtcagaatagcaggtacaaatatcctgggatagaattttgcttgatgtgttggaagaaaagcaaggaggccactgggttggaataggggggctgaaggggtgagtggttggaatgtctgatcagaggaaacatgggccttggagtaggcccttgtaaggagtgaaaggacagaatgagatggaaagccactggttaggtctcagcagagagtgaaagtctgactggctgctgcgtggagaaccctccacacaagatgcatgcgtgaagatcagttagggggccattgcaatagtctataggcaagagatgatggtgtcttagaccagggtgggggcagtggaggtgagaaggcatgattagagtctggatatattctgcagataaaaacagtatttgatgatggattggatgtgggacatgagagaaacagatgtgtccaaaatgactctgggcagctgggtaaatggaggtgcccttaattcacagtaaagactacaggatggtggtggtgtggggctgggagagaattcggtttgctttggatatctcaagtctgagatacctattagacagccaagtggagatgttgagtagagatgaaggcagtaagatttccaactctggtgctcagggcagagatttaggctgagacaaaaatttagaagtcaagatatatagatggtaaagtcacagaatcacctgggaaggggtgtaaacagaagaggtttgaggactaagcccaagaaagtcagagaatgaggagataggaatccagtagctgggtgcagtggctcacacctgtaatcccagcactttgtaaggccaacatgggcggattacttgaggtcaggagtttgaggccagcctggccaacgtggtgaaactcttgtctctactaaaaaaaaaaaaaaaaaaaattccagtaaaggaggcaattaaggaggagtagtgtcccagaaactaagagtgccttcagagtgattaccctgtgtcaaatgaaagctaagaactatgaaacgaccaacggatttagcagcagagattaggaggggcctgggagcctagtcctttgtggacgtttgggaggaatgacagggcaagctgggtcatccctgacatgtgggctctgagcctagaggcagagctgggttacagagtaggaatcccccaggaggcatggatttgcaacctggggtgagtggagaggagccagccagggcctactatgcctgacacggtggtattgggggtgaagagtcggtttcaggttggccaacttacctggactccacattcagtcacaaattgacttttgatgttgtttctaaatgcagttatacatatagtctttcggatttgtgttctttagaatgttaaaatatctaatttttataaccatgtttcttccatttttaaaataccaggaatcctgagaagcaggagtgatctatgccttaccctcagaacagggccaggacgagggtgaggcatgcttggtgtgtaaaatgcagaagttaaggcaagagcagggtctttgggaatgaatgcactttaggcacctctcttgccttacctgagtccaggccctacctacctcacatactgccaggtagtgtgccttcctttcattgtgggagcctccacattagtggtgggtcctggagaaggcagaccatagactggaggatctcctgttttgggtgggggcagacactactctgggagcccagaaaaaaagccaactgagaactatgcctttcagctaaaaagtgggtgcacactgccaccctgtggtcagccaggggaagattcttaggactgttcccaggattgcaccggcagaggtaagaccaagacacactccaggtcaccctgccctgaatttctcaaattgtggcattcgcagctgtgttggaagaaatcttattgaacatgttgatctctggcaatggagctcagtagtctgagttactgactcaaagagtgtcttaggtgattctaaggcacactcaagtatatagtccctactagtcaaaagccactgtcttgtcaggcatggtggcttgtttctgttatctcagctactctggagactgaggaaggatcacttgaggccaggagctggagatcagccggggcaacatagcaagaccccatttctaaaaaaaatttaaaaagctaaccaggcatggtggcatttgctactcataaggtcccagctactcataaggctaaggcaggaaaattgtttgggaccatgagttcaaggctgcagggagctatgatcatgtcactgtgccactgcactccagcctgggcaacagaggagaccccatctcttaaaaaaaaaaaaaaaaaatctaccatgaatcacacagcaagagagagatcttcctgaggtccccaggggtcagaggctttttccggctgctcagacacacaaaaagatggaacagatcaacaaatctttattaatagaaacagacagatggacagagaatgcagcaggggctgtgtgaaaactgtgatatgtgtgtgtgtgtgtgtgtgtgtatgtgttgtgtgtgtgtaaaaggccttctccaggccaggaagggccccttggaattaggcccacctgtctagatcctctcctcagtggtgggtcctgggctgaaaccagcttccacatcatacccccaaaggaagaatgggggtctttctggaatggaagacgctgacttcaagcattcttcaaattactctcgttccccagctgagctttgttgctgagggggttggggacaagccacaggggcacttgcatagcaggggagtagaatggggataaggcatatggtgaggacaggggagaaggggaagggcatcatagctgcaggtgaagggggcctccaaaaggcagctcctcttccccctcatcccctcaaattgctgctgggttgaggagtcagactgaggcagtataataccctcccccatccttaactctagaaccccggtttggtggggaggaggtgggaagctgtggagctatggaaaggcctcagttagtgagtcaagctgtgatgtgtgtgtctgaacacaactggctcccttggtataccgggggctccctctccagatgggtgtgagtgcatggtcctactgtacacacaggtctcagtatctatatgtgtctcatttgttcccatgggtctctgtgtttggatacataagcatggatatccctgctcatacagcaggaactcaggatctgcatggtgtatgtccctgtctgtaaacatgggctccagcaagtggatatgtgcgggcctgcccgctcctgtctatccgcaggtcttccttcaggcctggctggtcaagggtcctggccaaagaggtaggtggtgagctcaagccggaggccccgagcataggagcgaagagtatagaagagggtgaggaagtcctgggtgctgaagacagtgtcggccagcgcgaaggccagggtggatgggatgacgccccggccgtactccaccatccatgtgtttggcccccactccacagctgttgcctcaccaggcccgtgtactaccgtctcccctgggggacagggagcacccaagtgaaaagccagctctgccctgcccttccatggctgctgcttccctggcccatggactaactaggggtggggaatggagagggagcttcagaaaaacaaaaagccctacctcactgggctggcccagatggccactcctaaatgttctgtggcatcatctgacccacgtcctctctttggagaagccatggacggctacctcgcttggccatggacccactgtatggaagtctgcactcccaagcctcactcgaatcaagcagaaatttagctaaacctgggctgggcttgtagctaggaaaggggtagttccaggacttgtgaggctttatctgaaaagtgtagctgttcaaccaccatgcctgcgatgcatacacccaacctcaaatatactgggaagtccagtgctctatttcttaagtaaattgtttcactattaaaaaaaaaaaaaaaaaaaaaaggcaggggagcggccgggcgcggtggctcacacctgtaatcccagaactttgggaggccgaggcgggtggatcacgaggtcaggagatcgagaccatcctggctggtatggtgaaaccccgtctctactaaaaatacaaaaaaaattagccgggcgtggtggcgggcgcctgtagtcccagctactcgggaggctgaggcaggagaatggcgtgaacccgggaagcggagcttgcagtgagccgagatcgcgccactgcacgccagcctgggcgactgagcaagactccatctcaaaaaacaaacaaataaaaagataactgtctgtcttgtcttttggggtattgagagcatttctgattacccattgtctccccaacagttgttggaatactgtctgggcaaaattgtgcctgggcagataaaaatcagacatgtttaataggaagttgttatgttttgtttccccttgacagaggaaagcaagagtccctcagctctgccccaaccaatcacctgtggcttatgggagggtggaggaagggaaccatgaggagggcccccccaacacactccttttccatgccatgctccccgcaattgtgggcaccccccgcgtgaagggacccacttctctgacagagccttcttacccacctgggtagaagacctcacttttggtggtgccctctctccactggtggaaggtgccagagatgatggtatccgagatctcagcccagtagcgccctgagtgacaatcgcacatgacactatcagggtattcgcgccattgcatggcccagccaaacatcagaaaggagggcatgggcccctttacaaccccagcctcccagtctggcccgccgctagcactgacccgagtggccgcgggagcccaaggcggtgccgaagagcagcacatactcggacagcgaggcgtgcagaaggcacatggcgcccatccagccacccgcattcacgaacacccactgcagctcctcgtcgggcagcacgtggcctgggtgcagccgccgcagctccacgatcagacgagagaaggccagctcgtggtccagccctggcggaggcagaggggcggcggagtcagggctggcaccggtcctaggtccggggatggcgccttcggaaccctaggctccgctcccaggccggccgctcccctccctgccctctgcccgctcaccagcgtactgccgcgccaactgcgctatctcttcgcgctggaagacgaagctctgcgtacccagccagagccagacgacctgggtcagcaccgctgcgacagccaggagcagcgcggcccacgcccaccgccggcccacggcccactgcatcccggcgggcggcctggcacggcgcagctcaggagggagccggggcctgaggctttgcgctcacggcctcggagcccgccggctgcccacgaagcgctcccccgccaccgccgtggtacggctcgccctgaccaatcggagcggcccgggcaccttggcaccgcccttcggttgaaccatttcctcggggggagcacggaggagtggggcggtgccgcgcaagggaggcaagggggcggggccaagtcgcccagcggcagatgtcagggtcttcaggcgaggtctgacctagctagcgccactctacatacggataggtgtggggatagtgagacccagaatggacgggctcgcccgagctcactggctggtgaagcagacgagcccggaagtgaagccttctgggactcaacgattgttaaggcccgcctttaccaggactgaagcggagaggaggcagttcaagctgggagccccagggtgggcattccagaggcggtgacacctgagcagtgtctttaagagctcgcagaaggggcatgccagcccacagggaccagcccctagagcaaaggcctccgaggagagacagcttggggtcttcaggggcctacaattccttctctgttctgcggcacaaaatgtgagaggaggccttgtaaaccagctgagggttatcagcaagggaggaaggggattagattttgccaccgagtggcacaggactgtcgagtggaggggagcaagatgagtcagggagaccatgactgtgggccaggtaagagggaaggtctgtgtcagtggagatggaagaaagaaatgagcttaacaactctggaaatggagttggcagggcttgatctcttcttcctggaatactctttccccctttatcctatccccactttccccttatttggctaacatcctttgcatgtctagtgggcagttgagtgtccaggtctcgaagctcagctccagtctggagatagagctgggagttgtcattcattcagatactttgttggcacttgtatgtgccaagttctgtggtggccattagggatatggctagaaacaatggtcctactccccagcttagagatctcactggagagtctacagaaaacctctaagaatggatgaaatctcacagggagagagaatagagtaagataaggctaaagacagagccctggagagcaccaacagcaaagggtaggcagaggaagaagaaagcaaaaaggaaaagaaacaaaagagaaaagaggtagcattgtgcagtgattaagaaataaactctggagctggactgcctgagttcaaggcctggctcctgctacctactagttgtctgatcttgggcagtttacttaaaatctctgtaacttagttttcttacctgtaaaatggaatggagataagtacctacctcatagtacatatcttcattgtgatgagattagatgggttaatatttataaagcagttagtgcctcacatggtgagaattttatgtgatggttatatatataaaacataaaggaactggaaaaaagtagcctcatacatttttgaccttaaagaaagaaactgaggccgggcgaggtggctcataactgtaatcccaacactttgggaggctgaggcagacggaacacttgagtccaggtattcgagaccagccttggcaacatggtgaaaccccatctctgcaaaaagtacaaaaattagtcaggagtgggggcgcacacctgtagtcccagctactcaggaggccgaagtgggaggatcgcttgagcctgggaagtggaggttgcagttgagccaacagagggagactctgcctcaaaaaaaaaaaaaaaaaaaaaaaaaaaaagggccaggcacagtggcacacacctgtagtcccagcactttgggaggccaaggtgggcaggcagatttcctgagctcagaagttcgagaccaccctgggcaacatggtgaaacctcgtctctactaaaatacaaaaaattagccgggcgtggtggcaggtgcctgtagtcccagctactcgggaggctgaagcagaagaatcgcttgaacccagaaggtggatgttgcaatgagccaagatcgtgccactgcactccagcctggacaacagagggagaccctgtctctaaaaaaaaaagaaaagaaaaaggggccgggcactgtggctcacacctgcaatcccagcactttgggaggccgaggcgggtggatcacctgaggttgggaatttgagaccagcctaaccaacatggagaaaccctgtctctactaaaaatacaaaattagctgggcgtggtggcgcatgcttgtaatcccagctactcaggaggctgaggcaggagaatcacttgaacccaggaggcagaggttgcggtgagccgagatcacgccattgcactgcagcctcagtgtgaaacagcaaaactccgtctcaaaaaaaacaaaacaggctgggcggggtggctcacgcctgtaatcccagcgctttgggaggccgaggcaggtggatcacgtggtcaggagttcgagaccagcctgaccaacatggtgaaaccccatctctactaaaaaatacaaaaattagccgggcgcggtggcgcgtgccacccacttggcccagctacttggggggctgaggcaggagaatcgtttgaacccgggaggtggaggttgcagtgagtggagactgcaccattgcactccagcctgggcgacagagtgagactctgtctcaaaaaagaaaaacacacaaacaaaaacaacagtgaggaaaaaaaaaagctgaggcaaaattaatatagagagtttatttgggccaaggtagagaacgactgctcgggacacttttccaagttgccttggggaatgttgatacaagcattttttatttttatatgtggtttttatttttattttttattttcttggagatggattcttactctgttgcctaggctggaatgcagtagtgcaatcttggctcactgcaactcccgcctcccaggttcaagctatcctcctccctcagcctcccgagtagctgggaccacaggtgtgcaccaccatgcctggctaatttttgtagttttagtggagacaggtttttgccatgttggccaggctggtctctaactcctgacctcaggtgatccacactcctcggcctcccaaagtgtgggattacatgtgtgagccactgcacctggccactttattttaacttaatttaattttattttttttgagatggtgtctccactgtcgcccaggctggagtgcagtggcgcaatctcggctcactgcaacctctgcctcccagttcaagaaattctcctgcctcagcctcccgagtagctgggactacaggcacctgccaccacatccagctaatttttttgtatttttagtagagatggggtttcaccatgttggccaggctggtcttgaactctgacctcgtgatttgcccgcctcggcctcccaaagtgctgggattataggcgtgagccaccgcgcccggcctatttttattttttgagaccggatcttgctctgttacctaggctggagttcagtggcacgatcacagctcattgcagccttgacctcttgggctcaagggatcatcctgccttggcctcccaaagtgctggaattacaggtatgaaccatcatgcccagccacaagcaagtttttaaaggcaaaagaggacaaggagtgggctgacacaaaattgtttaacaggaattctcattggcttacagaaacaacattgattagtgattggctataccttgttgaactatagggtatgagttatggtgtctagactatggcattttatggctatgtggtatcaatttagagcccacatagcaagtggcttcaaggggtaattatttggctccatgggggtgggggaaggtgagatatgactgctgttacatgctgtcacttttttttttttttttttttgagactgagtcatactctgtcgcccaggctggagtgcagtggcgcaatctcggctcactgcaacctccgcctcccgggttgaagcgattctcctgcctccgccacccaagtagctggaattacaggcacctgccactatgctcagctttttgtatttttagtagtgacagggtttcaccataatggccaggctggtctcaaactcctgacctcaagtgatcccgcccgccttggtttcccagtgtgctgggattacaagtgtgagccactgcacccggcctttttttttctttttaaaattattattattattattattattattttgagacggagtctcttgcactgtcgccggggctggagtgcagtggcatgatcctggctcactgcaacctctgcctcccaggttcaagcaattctcctgcctcagcctcctgagtagctgggattacaggcgcccgccaccatgcccaactaattttttgtatttttagtagagacagggtttcaccatgttggccaggctggtctcgaactcctgacctcgtgattcgcccaccttggcctcccaaattgctgggattacaggcgtgagccaccgcgccaggcccatgctgtcacattttaatgcctctctgggcctgataattaaaagaggctggcatttctcagataaaaagtttcttttctttctcaagagtgatggaatgggccaggcacggtggctcacacctgtaatcccagcactttgggaggccgaggtaggggaatcacctgaggttgccagttcaagatccagcctgaccaacatggagaaaccccatctcttctaaaaatacaaaattagccaggcgtggtggcgcatacctgtaatcccagctactcgggaggctgaggcaggagaatcgcttgaacctgggaggcggaggttatggtgagctgagagcacgccattgcactccagtttgggcaatgagagcgaaactccatctaaaaaaattaaaaaaagagtgatggaatgaagggttaacacagtgaaatataatggtgagattcaatgagatgatgggaaatgatcattagacttctcagtaggggcatgtggcagagacaatgctatacattcacaaatccatttccttttgctcttggacagctagattacattccacagtctactctggatcatgtgattgcattctggccaatgaaatatagatagaaatgatgtatgacatttctagggctggctcctaaagtctcctctgaaattcttttttttttttttctctttctctttttaagagacaggattgtgctatgtcactcccagactggtatcaaactcttgggctcaagtgatctcccaccctggcttcccaagagctgggggactacaggcacatagtactatgcccagcttgaagttctttacattttctttcttcccatgtctattactgtagttatagaggatctctagtggagtattctgagaccctaggacatggcatagctactagatggatggcacctggatccctaaatactgcttggaacagagctccctgttacccccagaattgactgtagtgctaagccattgagttttgggagtgtttgttacagcaattaagattacttaccctgacaaatatagggcaaaagatgtgttacaatataccatgcagctaagagtagagaccagaaagatggcctctgtttttaagaaacttggcaggccgggcacggtggctcacgcctgtaatcccaatactttgggaggcagaggcaggcggatcacctgaggtcaggaatttgggaccagcctggccaacgtggtgaaaccctgtctctactacaaatacaaaaattagctgggtgtggtggtgcacgcctgtaatcccagctacaggggaggctgaggcaggagaatcacttgaatcaggaggcggatgttgcagtgagcctagattgcaccactgtactccagcctgggcgacagagcgagactccatctaaaaaaaaaaaaaaaaaaaaaaaaaaagaaacttggcagagaaattaaggataaagaaaggattatctagaggagaatacaggattgagggaacatgtttttgtattggttctggcttctgtggcattcccgactcgacaattcactaagaaccctgggcagcagggattagcccccaaatttggtctctgctccccattccaggaacagctttctgggatcatctgggcctacacctcccctatctggtctttcagaaagcttttccaggcctttccaggtgtggtgacttatgtctgtaatcccagcactttgggaaggccgagatggacagattacctgaggtcaggcgttcaagaccagcctggccaacatggtgaaactctgtctctaaaaaatacaaaaattagctagtcatgatggtgggtgcctgtaattccagctactcgggaggctgaggcaggagaatcacttgaacctgggaggcagaggttgcggtgagccgagatcaagccactgcactccagcctgggcaacagagtgaaactccgtcttaaaaaaaaaaaattatatatatatatatatatatatatatatatatatatatatatatatatatatatatataaatggataaccagcagccctcaggggtgctctgtctatggagtggccatttttttattccttcactttcctgataaacttgctttcacttcacactatggactcaccttgaattctttcttgtgtgagatccaagaatcctctcttggggtctggattgggatctctttactgtaacaacacctctgggatctggcagggagggagctggttaggcccatcttagtcattctcctcttcctccaaaaaatgaccttggggagtcatagaggagaattatggagatgttcctgaagagatgttcctgatgggggtcaggatttaattagcatgattcagggggcggcactgaagcactggtgttcttgggaaaatggagctgtctacaagctctagtcactcaaacaaatataaatgagagaatggaaaagcagatctcctgttcctgtctcacctctttgggtaatatgaacagccagtgggacaggtaaaacccctgtttctcagtcctcagctccctgccccctacctctgaaggtagcctcttcctattccagtctgaggaccctagggcagagatcaagtaaatgacccatgacctagtttttgcagctcaattgcaaaacacctggcttggcttggtcttgattgggggaagggatgtccctgctatggctcaacttctaactcactgggtctctgtcctgcattccactgaagcctcagaggcatctcacagcactgggaaaagccttggaaacttgcattcaaatctcagcttggccactaattctctgggtgaccttgagtcctctgggctccaatgtgaaatggggaagaggtttagacaatatttagatggctctttaaaagtactgggggccagctgtttgggaggctgaggcaggaggatcacttgaagtcaggagttcaagaccagcctggccaacatgtgaaaccccgtctctactaaaaatacataaattagcccagtgtggtggcacacagctctaattccagctacttggaggctgaggcaggagaatcgcttgaacctgggaggtggaggttgcagtgagccaagatcacgccactgcactccagcctgggtggcagagtgagactctctctcaaacaaacaaacaaacaaaagtaccaggggaggaattaatttgaattttatcctagtgttagccaattggtcccatccaaggaaaatttagaaaagggaaggggatgtgtaaaggaaacactaggccccacctagatggtggctggagcttctgatagtcctgtactctccacatttttagactttcttgtacttttttttttttttttgtgacggagtctgctctgtcgccaggctagagtgcatgggcacaatctcggctcactgcaacctctgcctcctgggttcaagtgattctcctgcctcagcctcccgagtaggtgggactacaggtgcacgccaccaagaccagctaattttttttttttttttttttactagagacggggtttcaccatgttggccaggatggtctcaatttcttgaccttgtgatccgcctgccttggtctcccaaagtgttgggattacaggcgtgagccaccgagcccggccatttttagatttttttttatagcccttttacccctcccacttcttgtgctgtcttggtgtgtgtatgaaaaagagagagagagagagaggaaggagagcaagagtgagagaaaaagagaatatggttacttagccctttttcccaaatctgtttttgcttttggggataggatatggttatggtgaagtactccacacagaccccaccaagtttgcctctcctgcgctacagccactgagcaggtgactgaggcgctgttgatccagggtcatatccttactcgccctagaatgtaagctcaggcagggcagaggccatgcctgacatgtgcatcccaatgctttacacagcctaggtgcctagcacatgctagatgcttggtaaatatttgctgaatgaaagatcaaatgaatgattgcagcaagcaagtcctgtaggcatcctggagcccaaggattctgcagtaggcagctttcacagaggttcttccagtgtagtggctctagctctgggtgaagtaggatcatcaatgtcggcccccagggttcacagctgttctgagccccgcccccaggtggcagggcagcccagtcagtcagtcacgtgctggcggctggccaatcatcgggggcggcgcggggaggggtggtgtggacggagaaagtgaaaggtgaggcacggccctgcagattttccagcggatcccccggtggcctcatgtcgcgcagtggaaccgatcctcagcaacgccagcaggcgtcagaggcggacgccgcagcagcaaccttccgggcaaacggtaactgcaccgcggcagggactcgctggggcgcggagccgagccctccccttccttaggaagctttcgtcccctccgaaggttggaacgctcatcccgagccagaccgacaaggcgtacagtctgcaggcctgtacgagcagcaggccaattggcgctgggaaagtccaatcctgggcctctagctcctgagcgggacagggccgagagggcgctcccgagcttgggcctgctggtgggtgagacccaggagagagggagctagagagctctgaggactgatcttgactgtctgcccccagaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtaagcctgtagagccctgcatctgcaggctgggccacggggagtagttccctcttagaactgtcctccacccacagggatagtgaacctccttctgggtcatatcccaccaagctttttggtcccctagggtgggccttccctactcccttgtagcctgtccagtctttgaagcccaccaggtaactggtggtatggggcagtgagtgcttctagcctatccttgtcggtaggtgaatccccagtacgatagcaccttcctgtttgacaacgacttcccagctctgcagcctgatgcccccagtccaggtaacctggctccaactgctgctggggaggagggtggctagacctcttgagggacttctgctgcagagagtgatactcctttacctcaggacccagtgatcatccccttttccaagcaaagtctgctcgaggagtctggtaactatggatttcccctcttacaactttcaaaccagagttggagactcagcattggggttcggccctgcccgtagcacagccaagccctacctctcggttatcttttctcccgtcaccacccagtaaggtcatgtgcttccacccctggtcggatgtaacgctgccactcatgtcggtccctgagatccgggctgttgttgatgcatgggcctcagtcacagaggagctgggtgcccagtacccttgggtgcaggtttgtgaggtcgccccttcccctggatgggcagggagggggtgatgaagctttggttctggggagtaacatttctgtttccacagggtgtggtcaggagggagttgacttggtgtcttttggctaacagagctccgtatccctatctgatagatctttgaaaacaaaggtgccatgatgggctgttctaacccccacccccactgccaggtaagggtgtcaggggctccagtgggtttcttggctgagtctgagccagcactgtggacatgggaacaggattaatggatgggacagaggaaatatgccaatgatgtggaggcttggaggtaaaggacctgcctgttcttctctgcttttgccccttgacaggtatgggccagcagtttcctgccagatattgcccagcgtgaggagcgatctcagcaggcctataagagtcagcatggagagcccctgctaatggagtacagccgccaggagctactcaggaaggtgggagagagccaagccctgtgtccccaaggagtccctaactttcttatcccatgagagaggtgtgtaaaggagaaagctagaggtgaactagtagagagagacttgctaggaggccttagcaataatccagtaatctaaaggaaagatgatggtgacttagactcgggtggttagtggtagaggtggtgagaagacatcagatcctgggcacattcttttcttctgcttcccttgcctatttgctgaccacactccggctcctatgtcaccttgatgacttcctatccattctgtcttcctaggaacgtctggtcctaaccagtgagcactggttagtactggtccccttctgggcaacatggccctaccagacactgctgctgccccgtcggcatgtgcggcggctacctgagctgacccctgctgagcgtgatggtcagtctcccaagtaggatcctggggctaggcactggatggaggttgctcccagtagggtcagcatctggaccccaggctgagagtcaggctctgattccagatctagcctccatcatgaagaagctcttgaccaagtatgacaacctctttgagacgtcctttccctactccatgggctggcatggtgaggcttttcaagtacctatatttagccccaacaccatttctgggctcctggggctcagcctagtgaactgcaacctcaaaggagcaagccttgaaacagttgctgggggaagtggccagagtagagatgctgggactgagggtggagcagcaaacttggtgaaactacatctccaatgtgctttctaatctcctgccagctcttctcaagcaggggatcctgggagatgtagttttcagatacctggttgggtttgggagtaggtgctaacctggataactgtaaaagggctctctctccccactgtctctcttctttctgtcaggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggtcaggactcagaacagtctggcgtctccagactctcacatgcagtatgtgcaggcacctgatacttctgttgcccttgtgctccagtcattgcacaaggcagaaaacagctctggcaggaagggactgccaaagttaggagccctagggcctggaaggagagtatggtcctcagatcccccttctctcctgcttcctccagggaacccaacagtcatgaccctgatagtttcccataacaacctgggcattccttgggactcaggagctgctaaactctttcatcccctggtggcttcagcagtccttatcaccagcctcacaatcccacaggcccacccccagtgggcctgtggcattcatatttcatattcatatttcaaaccacaatatccagcaaaatgtctcctgagcacccagaactccataccatcggccgggtgtggtggctcatgccttaatcccagcactttgggaggtcaagatgggaggattgcttgagcccagaagttcgagactagcctgggaaacataggaagccctcgtctctacaaaaaaaatttaaaaagttagccaggtatggtggcatatacttgtggtcccagatacttgggaggctgagataggatcacttgggcccaggagtttgaggctgcagtgagccatcatcatggcatcattgcattccagcctgggcaacagagcaagacctcgtctcaaaaaaaaaaaaaaaatgaagtccatgccaccattcttggcagcccagcccttatcctccttaattgctccctgtcccttttccaggctgcagagagactaagggcacttcctgaggttcattaccacctggggcagaaggacagggagacagcaaccatcgcctgaccacgccgaccacagggccttgaatccttttttgttttcaacagtcttgctgaattaagcagaaagggccttgaatcctggcctggaatttgggcagatatagcattaataaaactgtgcatctcaaacttttatcacatactctaatatcagaggagtgtgaaccttcagagatctagggttaaaagctaaaggcatagctccagctacaacttttctttgtctacagatgtgtaaaatcttgatgcaaaaatctaccccaaatttctgaacaaaattaatattcagtattatatctagcctatagattctcttactcttggtgtaatgacagcattcccagtttaggaaactgttttaaaatcttgtgtcttggttgtggctgaggctttgggaaggccagagccccctttgccaccacccctagggtgtcagctccaagcctgggcctgagggtttgatggggagcgggtgagctggatcctgatcccagtggtcagtggccagatcctgggctgctggccagaaccctggctttgtgagtggctgcagcttggctctgctccagcccacaggggctaaaggggcgggtagggagtgtgtagggagcgtgtacgtggctttgtgtccttggctcctccagactctcacatgtgcgggcaccatatacttctgctgcccttgtgccccaatcattgcacaaacagaaacagctctgacagagaagggactgctagagttaggaaccctagggcctggaaggaaggtatggctctcagagtgacctggggtctattcagtcccctctggacctgtttccctggcaacgctgtgggaacaacagtctatgcaacacaggagacctgatgccactggtgtcctccaaattttaaggcgaaggcaggcattgtgaatgatgcagggatggccagaatggtttttgccttcgttctttccccaggggaaccaagcctgtgggtggggaaatcacactaaggctggacagtgactgaatctggcccaggagaaacttggctgcctgccttccagaccagggtttttcttccctgagatcctggaggggaagtacctggaacttgggagtgttgccctggtctggaagatctgatagacatagtatgcacacatttccctcgagcttcccatgactggggagtagatgagaaccctgagtgggtggctggtttcttgaggcatacatatacccgacacaccttacacctctgatttggctgcattttggaatcatctgggaagttttaaacaatactggtgcctaggtccaacctccagagactctgatttaattggtatggggtggggttttgcagttttcaaaccacaatatccagcaaagtttgagaaccactgccctacacagccctccttcttctttatcttatctggagtcaggggcaccttcagcttctccaatcacctggccagtggctcttgtttccagtcctgctgttccagggtggataggccttcaggcggagacccaggaatccagctccagactctgtcccaagcagcctcttcagtccgggacgggacaggaagtgtccggcggaggtgggggtggaggtgattggcagttggagtctccaatgggccaggagcccccctatttcactcctctccttccattggcgcctgggcggagccacccccgctgcgggggcccagggcgggagggagtagaggccggggcagagggcgagggcgagggcagagggcgctggcggcagcggccgcggaaggtgaggcctggtgtagacgccaaagtcttgtcaggagtgggcggggcagggtgatgtatgatctgaaaggccgggagtgactaacgactgtcagtgtgtatgtgcctgtgaggctgtgtctgtgtgacagtatcattgtgtgcgattctgagtgtcacttggtgcagctgtcagctaatgtgactgcgtgtgtcagtgtgcatggagctggaggtgcatgtggctgtgcaagtgtgggattatgtgtgtatctgtcaatgcacatttttggtgggactgggttgtgtgtttgtgtgagagggactgtgaccacatgagggaatgtgttttgggaaggtctctgtggggtttgagaccatgtctgccagcgtgagagtgggtggctggctttgtgtgtgccaatgtgtgacttcagcagtagcagagcaaaagtgaggtttgtgactatgtgtgtgtcctgggtcctaacttaaaccgtgtgggagtacagctgtattcatgtattgggggacctacctcaagagcccatgggactgacctttctcttaagtgtccagcattgacagacacacccacaacacacgtggcttcccctccttcctctgctcaagaatccccagcatcaggtctactgctccttattttctgtttccctgcttggctcagtttcccttacgtgccgttttctgtccctattgctgtcttactagagatacctctgtgtgtttcttcacagctttgttcccctggtctctctgtgtgggtgcatttccagttttgcctttcctttctctctgtctctctctgtgcccctatgcctgccttccctgctgatcttgatctctggctagttgggggcctgtaacagtgatgagatgcgggtctgccccggctaagagctccctgagagagggggcttagaccatgatgaagagttgcttttaggcggtgctgtgagccctggtaggggctgtgtgcgtgtgtgactgcgtgagtgtatgaatatgtgtaagtgtgtctgagtctgaggcggtgtggctgtgcccgtgtgactgtgagtgaccgcatgtgagagtgtatgagtctgagggtaactgtgggtcttgtgtatctggcaccaagggaccaaaggcatttcctggtgctggggcccaaagaatggagggggaggtgagaaatggctggaacttccaggggagggggaactggactctttggcctctccttatatcccccaacctggggcctcccttcccctggaagcacagccagctgtaccctaaggaaacatttggtgaggagtgaaaagagggggagtttgtatctgatttttcatttgattgtgtccacgtgtgttcaaaggcatgcccttttgttaactgtgcctcttgagtctgtgtgtgaatgtgactgtgtctggctgtgtgaaaacacagggcatctctcactgacagggacataagtattagccaccctcctttccctcttgctggccttggctgtgtctgtggcttcctttccagacttgggggaggcctcttcccttcccagggccttgggtgggagggaaggagggaggaggcaggagcagggagggggcctggctaggggcggcagagctccagggcttagagggggcggggcagggaaggggaaggaggggatggctctggggtcccagaactgagtggagtaggagacgggggctgtagctggtgagaggaagtcctagaggctatggacactctgctgctgggatcaccgaggtagggtggggctaagcagggtaagtgtggctgggtggccaggggtgagccaactggacacactcagacagacacacagacaggtggaggccctgctctgctctactcctactcctgaagggtccgtgctcttcctcttctggtcttgttcccagagccctttccccgcctgccccttcgttctccatccacttcaccactgccttcctccttctgctcccccttgtccagaatctctctctcctagcatcccccacctgccatatagacacactgttagctttttgagtttccgctgaccccaatccagtctttccaacacttcccacactgccccatcacctcctagtcctgatctctgagtatctcttggtgctgtggcccccatgcctgtccctgcaggctcccagttcctctcagcctctcatccccttccctcccctcccctccccgccaccctgtctccatttccatctgctggctcctcgcccacccccagcctctggcagcagccagggcatctggatctgcttaactacacagccccagcctgcaccctagccccatccagcttcacaaactggagaccaacgaagtgtcaagagccaggcccagctgagtggcccaagtagccagaccaaggagccaggttcaggcgagaagcctggcagccagggcaggggtgggcctcagggtgggagtgcaggatgggctcagatccatgatgacacccttcccccagggtgataaggtctgcctaggttaatcagaggcagtgataagccctggaccaggtgggggtaaataccagaattcccaacagctggactggaggggttaatgggagtggctgagctggtgccagtgcttggtgccaggggtgggcgccaagggcagtggagggggagttgctggcacagtctgttgcctccggcttttgttctgggccctaagcccaggactgagatggagggtgtgagggggtgtgtgtgtccgtgtgtgtgtgtgtgtgtgtgtgcgcgcgcacgcacatgcaaagcactgggtatacagtgggaaaggggacctcaggtcagttcccgcagtgatttctaacagccttaccccacttggtgcatcaatttttctcctaggaagcctcagttttggagaggaagagccaggctttagcctcccatctcaggggtcggggatttttgactctacctctccccacagatgagcagcagctgctcagggctgagcagggtcctggtggccgtggctacagccctggtgtctgcctcctccccctgcccccaggcctggggccccccaggtgagaagaaccctgctctacaacctcccaactctgacttgtgacttgccaccctcactgtggccccttcccatgcctaacttgtgacaccacccctgctcctgtcatccaacctgggtcctctctctaccctgttctctgacctctgtctccagattataacatgatgctttgatccagtgagccccccaccacccaggttttaacagtctgcttcttatctgtcattctcacaaagtggggaggggggtaaaggaagagccttacctcagaagtgccctccacaggggtccagtatgggcagccaggcaggtccgtgaagctgtgttgtcctggagtgactgccgggtaagtgccccacctgcctgttggtctgacactcatgaccctttcttgactctgcctagtcctcatgtcccgcccctcccatccttgcccgctctgtccgtaatcctcacctctctatctgtcctaaccgtctaactatccttattcaacagaaaattcaactggtatgttttgagcatctgccaagtgccaggtctattctaggctctgggatacagcagtgctaatgccaggcaaggtgcctgccttcaaagagcttacatcttagtggggaagacaataagcaaaggagaatcttgccagatagtaatacctgcagggcagaaaaaccaaagtgagggatgtcgaagaggacaactgagtggttactttttttttttttttttccgagagtctcactctgttgcccaggctggagtgcagtggtgcaatctgggctcactgcaacctctgcctcctgggttcaagcaattctcctgcctcagcctcccaagtagctgggattacaggtatgcgccaccaggcccggctaatttttttttgtatttttagtagagacggggttttaccatgttggtcaggctttgaactcctgacttcaagtaatccacccacctcagcctcccaaagtgctagattacaggcgtgagccaccgtgcctggcccgtggttaccttttaattggtagttaggaaaggtctctctgaggagatggtgttgaagctgatttctgaaggtcaggaaggaggcagccatgcacacattaaggtataagcgctcgtgacagagggagcagctagtgcaaaatgtcctgaggtgggaatgagctcaatacccccgaggaatagaaagaagaccaatgtagctggagttttcagtgggtgagggggagagagataggagctgaagaaggaaaggaaggcaagggctggctcacaaggggctttgaaagccagtaaaaggaatttgggttctattgcaaatgcagttagaagccaatggagagctgtggcatggtctgacttatgcttgacatcttgtctttgtccattgtcacctcatctcacttccagggacccagtgtcctggtttcgggatggggagccaaagctgctccagggacctgactctgggctagggcatgaactggtcctggcccaggcagacagcactgatgagggcacctacatctgccagaccctggatggtgcacttgggggcacagtgaccctgcagctgggctgtgagttggggagggtggcactgatgacacatagggatcctgagggagtatgggacctaagtaagccctctgggaccaggaggacctgaggactgctcagtctccaggtgtaccctctgcctctagaccctccagcccgccctgttgtctcctgccaagcagccgactatgagaacttctcttgcacttggagtcccagccagatcagcggtttacccacccgctacctcacctcctacaggtgtgtgtgtgattgggtgtggatgcctacacatttgggtatgctggtgcaatgtgggtgtgggaggcagggaggtaggttctgaggaaagcctcagagagggcagaaggccctcttttccttcctgacttcagatggccccctccccaccaggaagaagacagtcctaggagctgatagccagaggtaggacgtgagggagcatgtgggggctccagctgggtcccacagtaggcattttcaaagccaaagaccacatcctcccttctaggaggagtccatccacagggccctggccatgcccacaggatcccctaggggctgcccgctgtgttgtccacggggctgagttctggagccagtaccggattaatgtgactgaggtgaacccactgggtgccagcacacgcctgctggatgtgagcttgcagagcatctgtgagtacccaccccagacctcccccagacccccatcacagagaccccaaatggactcagatctctccactcccagagttcccagtttcctcccaacctagactccatttcacacagagaaggccctgtggagcctgagaaggcaaagggatcagagcccaggaggcatggttatttcagcacacactctagaggctgctgctgctacccacactgcctgattggctgcctagcctcagagcaggccctgtaccgaatgcagccatatgtcatgtcctcccttgctggcccctccttccttgacctgtgcatgggagtgggacactaaaactctagctggaatttgggccaattcatcttccttggatgaaaataggaattgctcttttattcactcaagaagcacttcaaaagtccacccgcagatctcaggtggtttctttttttctttctcttcctttttttgttgagatagggtctcactttgtgcccaggctggatgaagttcagtggtgtgatcttggctcactgaagcctgcaactcctgggctcaagctatcccctcacctcagcctcccaagtagctgggattacaggcatgtgccaccatgtctggttaatttttaaaattttttgtagagacagggttttgctacgttgcccacgctggtctcaaactcgtggcatcaagtgatcctcccgcctcagtcttccagagtgctgagattacaggcatgagccaccacacgcggcctcagcccatttcataatacagatgaccggtctgagtctaatggatgatcaagtttaagatttcccctcccctctcaggagtgtctggctaaggctcctttaaacacacactttgggaagtggtggggaagtgatggagacccatagcctaccctgactttgtgtcttgatgcccttagtgcgccctgacccaccccagggcctgcgggtagagtcagtaccaggttacccccgacgcctgcgagccagctggacataccctgcctcctggccgtgccagccccacttcctgctcaagttccgtttgcagtaccgtccggcgcagcatccagcctggtccacggtgaggcctggagtgcgtcccaacccacggctgtgggtcctgtctctgatttcacgatcctgggtgttctgtatagctttccagtgctggcaggtcactgaagacccaacacttccctgtgggccaggctttgtactgggtgctgggttgcagtggtgactgagagactgaatgtctgtcctgtggggctataactatgaggtaaagcacatctgtgggaagatagcaatggcttttaaaaaaataactttttttttttgagagggagtctcgctctgtcacctaggctggagtgcagtggtgcaatctcggctaactgcaacctccacctcctgggttcaagcaattcttctgtctcagcctcccgagtagctgggactacaggtgcccgccgccatgcctggctaatttttgtatttttagtagagatggggtttcaccatattgcccaggctggtctcgaactcatgacctcacgatctgcccacctcggcctcccaaagtgctgggattacaggtgtgagccacggcgcccggcctaaaaataactcttaggaaaaggagtgcctttcacctagaggcacattctcctacaaacactcatatacccagctgaggatagcagtgaatttgtgtctcaggagatgttaaagattggctggtatcaggactagggagcagcagcatcagataataactaatatttacttagtgctgcctgcctgtggggtacctggctacactttttgcatacattgttttgtttaggtagattcacctctattttacaaatgaagaaactgagtcacagagaggcccaaagggtcattcaatatgtggttcaccaaagatttgaacccaggctgcctgatgccacagcctctctaagtaatcactactgccgtcttacaaacactaggtgtagactcagctccaccagacacccaacatgagacctagacagctccactaggctttaggatgagactgggagcaaggggtgctctttgtagatggtggctgggaaggccctgcacttacaagctgggtaacagtgagtcatgtttacccccaggtggagccagctggactggaggaggtgatcacagatgctgtggctgggctgccccatgctgtacgagtcagtgcccgggactttctagatgctggcacctggagcacctggagcccggaggcctggggaactccgagcactggtgagagacaaagccaaagaaaagggcagaggccccatcccaaagcatctatcagctgaaccagttccaaaagacaggcaggtggcacatgccctgcccacacaggcacggcaattgctgtcccagacttcagacttgtttgcctacagccctgtcttagcttaagtcctgccccttggcctccacccctgcagttacaggctccacttctacaagcttggagcttgccatgctccctaggagctggccatgccctatcaggctcctcctcctaggtaccttgacttccaacctaggccttggccttgagcacaggacgtgacccccgtccccaccagcgtatggacacttattgggtcttgccttcctttagggaccataccaaaggagataccagcatggggccagctacacacgcagccagaggtggagcctcaggtggacagccctgctcctccaaggccctccctccaaccacaccctcggctacttggtgagcttgggcagatgggcaagacttgtaaaggactgaaaaccccagagaaaaggggacaccgagcttggtcaggcttgctctcagtgacatgtggccctccccctcagatcacagggactctgtggagcaggtagctgtgctggcgtctttgggaatcctttctttcctgggactggtggctggggccctggcactggggctctggtaagtgactgccattggtccctcagcctctgatcctcacacatgctctgatgcccatagaccacattcatctccacccttcatgactgcccgctgaacctgtctgattctggaactacctccccatacctccatcccccatgccccacttgattttaactgattcctctcctgaccctttactaataaaccctttggcggagactgagataacccacattgttggagagacagctgcctttctatgccccaggctgaggctgagacggggtgggaaggatggatccccaaagcctgggttcttggcctcagtgattccagtggacaggcgtccaggtgagtaggacatccagaagatttggacttggagatgtttgcccctattttgagtgtccagattaagagctggctgccctagtcattttaaaacatgctgggaatccaagttgggtctcctcattttaatgatgtctaggctgagggctgggcctttcattcttgagtccctgggctcagaagtgggtctctttccctcctctcagggtactgaggaaggaccccaggtggacttcccttcagggtattagtattagtactaatactcttccagggtattagactcagagctggatattcatccttttttttcaaggtgtcttggatcagtgctaggtcccctccttcattcttaatgtgcctgggcccagagctaggtccactcccgtatcttcagtgtgttgaagaggctccctcagagctgggctcctctactcatcttcagagtgttaagcttagaactgagtcacctccctcattctcagggtgtctgggccaagatccggtcttactgtctctcctgatttgcctcctgcttcttctaggagctccaaacctgtagaggacccaggagggcttcggcagattccacctataattctgtcttgctggtgtggatagaaaccaggcaggacagtagatccctatggttggatctcagctggaagttctgtttggagcccatttctgtgagaccctgtatttcaaatttgcagctgaaaggtgcttgtacctctgatttcaccccagagttggagttctgctcaaggaacgtgtgtaatgtgtacatctgtgtccatgtgtgaccatgtgtctgtgaggcagggaacatgtattctctgcatgcatgtatgtaggtgcctggggagtgtgtgtgggtccttggctcttggcctttccccttgcaggggttgtgcaggtgtgaataaagagaataaggaagttcttggagattatactcagaaattattatccaatgctgctttattatttggctattgggggcttcagcccattttccttagcatcccaaaattcagcttgggcagagtcccatggagctttctctcttggtgctcaaaccactgtgacaggctggggttctgggggtggatgcagatgctgcgttgagccaggtgaagcctgggtagagaaaggtccaaagctatgaccatagcttggggatgagattagggtcatccccaatgaccctaattaggggttaggagcaaggaccagatgaaaacctgagttggcaacagaattagcagttagggctggggttgggatcagcattaggggttgggactgaggtcagagtcaggggtatcaggggtgggagctcacacgaaagcctggaggtgacagtccccgtcagcctcctgcagttccacctggatgaccttcctcagtagcttgtctgagagtggctttcggtagagctgagtacagcaggcagtgctgggtggcagtaggaatgctaggggcaacagggccatgtgttcagagggatcctgaccaagaacctctctgccagacagctggaggttaggttggatttcctgggtcttttcaacttcatcccattcacccagacccccagctttctatacccccaagtactcacctgctgtagggtctgggctcaggagcaatgacagcagcaggaggctgcagaaggttgggggccccttcatgttgctcagcctagactcttccttctctctctctcctcctcccctaccttttatctgggtgccttttacctggggcactaggtgagtcagaggcagttggagatgtactgtgtaacctgtgacactcctgggccctaattctcggcctgttgccacagactccttcctcatctaggatagccatttcccatgtgctcctaagctctgggcagtgaagctgggggaaggattagtgtgtgatgtagggactgatggaacagtgagtaagggcagtcaagcatccacagcagcatctaatgatagacattcacactcaaatacgcatagccacacattcaaagtcactcctcaatcacttggtcaacctcacaaagtcatactcatgatcagacaaccgtagacaaaattatatagccccggttatactcaaagtccctctcccacccaattgtgttttcacagtcatccaatcacaagcaaattcacactagcacacacagattctgacaggggagttgtgggtggggtgggagcagagggcaggcaagcaccaacttgccttcaccttctcctccccacttcctggcttggcctgcctgtcttatcagaagaaatggatgggaatgggtccctagagcaggcccttctggctgaggagggttcttggctgaggccagccccattcagagaagctgggggaaggccctttatggtaatattaagggtgttttggagatgctgaggtggggcagctttccccaggatgatgagacagggaggaaaggtggggtagataaggagggaagataaaagctctttggtgctaggttactgttgtcaaaacaggtaaaaggcccagtgaatcttaccttcctgtttttgtctagtcctgttccaggaatgccaacctcttgccagcaggggcctagggtctccagatccagtccctgacttgttgaagacattggactcaagcactcaattacatctttgttttactcaaattccagcctccttcctgggcctggaggaacatcccctctccctacccgggttcagtcacaccctcatgtccaactgctataccccctgcatcccaccctccagctatagccgcttctctcctaatcccatacccagctgtcactccgttctttaccccctccctgcaatgcaaagctcctattcatctttatccagtcctttccaggaagctgggagctgcctagggtgggggtatgctgccaagttcagtgcccctggctcctttaagaatttcccactcctggtccagggatctcaatctgcaagtcctgctctatatttgaaacataaaccaagtcataacacagtaatccagcccttatcttgttgcagtcgtctgctgtagggtcagccactctctatggagtaggctttctcttgggatagggcttaggcctttctcttgtgtcccaaggcttgttgggtgtagccactgaggaaatcccaaggaaatctcccaggttcctcgggcttgtcatagaccttcttcatcacttggctgtgcgctacatactttctggtcttccctgggcttgtcggtccagaagtcttcggaaggcagaaaacaaatgcttttgggtgtgcacccatctctcccctttaagaccaagtcttctccccagccacaatctgctgtagctttggccccagccagaaactgggccatctctttctttaacttaatcttgtggtgccctttctggtgctgggtccagtccctaactgaaggcaggcccacatagcagtccctgattctcaccacttgaggagttagtgggtcccaggctgattgggtgacagaagggggcggcaaagagcagtggcccttggactctcctagggagggagcaagaacttctaggtaggctgatgccaaggatctctaaacttgggatcagactggaggagccaaaagttggggtatgaactgtaggaaaggtcgccaacccaagtcgctcctctgtggcaggcagcttctaggaatctccccctcttaccatttacagcctgcatcctacacctctgcttaaaatgttttagatatactcccagcagagaa

SEQ ID NO: 4 - DNA, 94 bp, Homo sapiens
tttaaactgaggtccttggctagaatcctcagatttcatatggagttttctagaagtttttgaaatttctttatgaatctcccttgcagtcttg

SEQ ID NO: 5 - DNA, 17 bp, Artificial sequence
  27-486/30/A OLIGO MIS sequence
ggaatctccccctctta

SEQ ID NO: 6 - DNA, 20 bp, Artificial sequence
  27-486/30/A primer PCR PU
aggcagcttctaggaatctc

SEQ ID NO: 7 - DNA, 20 bp, Artificial sequence
  27-486/30/A primer PCR RP
ttctctgctgggagtatatc

SEQ ID NO: 8 - DNA, 19 bp, Artificial sequence
  27-417/43/A OLIGO MIS sequence
aatgcctccccagacctca

SEQ ID NO: 9 - DNA, 20 bp, Artificial sequence
  27-417/43/A primer PCR PU
agaccttactgctcccatac

SEQ ID NO: 10 - DNA, 20 bp, Artificial sequence
  27-417/43/A primer PCR RP
gggcgctaagatatagcaac

SEQ ID NO: 11 - DNA, 19 bp, Artificial sequence
  27-180/28/B OLIGO MIS sequence
tgggtgccttctggttgga

SEQ ID NO: 12 - DNA, 20 bp, Artificial sequence
  27-180/28/B primer PCR PU
ctcctcctcaaaccaactag

SEQ ID NO: 13 - DNA, 20 bp, Artificial sequence
  27-180/28/B primer PCR RP
tgagagcagagaaaaggtcc

SEQ ID NO: 14 - DNA, 18 bp, Artificial sequence
  27-484/27/A OLIGO MIS sequence
gaggtccttggctagaat

SEQ ID NO: 15 - DNA, 20 bp, Artificial sequence
  27-484/27/A primer PCR PU
tttaaactgaggtccttggc

SEQ ID NO: 16 - DNA, 20 bp, Artificial sequence
  27-484/27/A primer PCR RP
caagactgcaagggagattc

SEQ ID NO: 17 - DNA, 19 bp, Artificial sequence
  M1 OLIGO MIS sequence
agttatctgactggagaaa

SEQ ID NO: 18 - DNA, 20 bp, Artificial sequence
  M1 Primer PCR PU
gacaaagtctcagggatcac

SEQ ID NO: 19 - DNA, 20 bp, Artificial sequence
  M1 Primer PCR RP
aaatttcctgcaagtgcccc

SEQ ID NO: 20 - DNA, 17 bp, Artificial sequence
  M5 OLIGO MIS sequence
cgagggcccgtcttggg

SEQ ID NO: 21 - DNA, 20 bp, Artificial sequence
  M5 Primer PCR PU
ctacaggctaaggactcttc

SEQ ID NO: 22 - DNA, 20 bp, Artificial sequence
  M5 Primer PCR RP
ggtagtgacttgtgttcagc

SEQ ID NO: 23 - DNA, 20 bp, Artificial sequence
  M6 OLIGO MIS sequence
acacacagggatgttgacag

SEQ ID NO: 24 - DNA, 20 bp, Artificial sequence
  M6 Primer PCR PU
gacaatgacacacagggatg

SEQ ID NO: 25 - DNA, 20 bp, Artificial sequence
  M6 Primer PCR RP
atcattgccgcgtttgttgg

SEQ ID NO: 26 - DNA, 20 bp, Artificial sequence
  M7 OLIGO MIS sequence
catcagactggtactgcctc

SEQ ID NO: 27 - DNA, 20 bp, Artificial sequence
  M7 Primer PCR PU
cataccattctgcttcctgc

SEQ ID NO: 28 - DNA, 20 bp, Artificial sequence
  M7 Primer PCR RP
atctgtgagccttgggaaac

SEQ ID NO: 29 - DNA, 19 bp, Artificial sequence
  M8 OLIGO MIS sequence
aggtatggctgtgaggttt

SEQ ID NO: 30 - DNA, 20 bp, Artificial sequence
  M8 Primer PCR PU
tgaggagaagcatacactgg

SEQ ID NO: 31 - DNA, 21 bp, Artificial sequence
  M8 Primer PCR RP
aacttcaccaatatttctggg

(19) United States
(12) Patent Application Publication
(10) Pub. No.: US 2008/0254451 A1
(43) Pub. Date: Oct. 16, 2008

(54) Compositions and Methods for Treating Schizophrenia and Related Disorders

(75) Inventors: Ilya Chumakov, Vaux-le-Penil (FR); Daniel Cohen, Le Vesinet (FR); Fabio Macciardi, Paris (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: ARES TRADING S.A., Aubonne (CH)

(21) Appl. No.: 11/575,844

(22) PCT Filed: Dec. 12, 2005
(86) PCT No.: PCT/EP05/56677

§ 371 Date: Jun. 29, 2007

(30) Foreign Application Priority Data
Dec. 28, 2004 (EP) 04293143.6

(51) Int. Cl. C12Q 1/68; C12Q 1/02; G01N 33/566; G01N 33/53

(52) U.S. Cl. 435/6; 435/29; 436/501; 435/7.2

(57) ABSTRACT

The present invention relates, generally, to methods and compositions for detecting or treating mental disorders, such as schizophrenia. The present invention more particularly discloses the identification of human genes which can be used for the diagnosis, prevention and treatment of schizophrenia and related disorders, as well as for the screening of therapeutically active drugs. The invention further discloses specific polymorphisms or alleles of the CNTFR gene that are related to schizophrenia, as well as diagnostic tools and kits based on these markers. The invention can be used in the diagnosis of or predisposition to, detection, prevention and/or treatment of schizophrenia and related disorders.

### FIELD OF THE INVENTION

The present invention relates, generally, to methods and compositions for detecting or treating mental disorders, such as schizophrenia. The present invention more particularly discloses the identification of the human CNTFR gene, which can be used for the diagnosis, prevention and treatment of schizophrenia and related disorders, as well as for the screening of therapeutically active drugs. The invention further discloses specific polymorphisms or alleles of the CNTFR gene that are related to schizophrenia, as well as diagnostic tools and kits based on these markers. The invention can be used in the diagnosis or detection of the presence, risk or predisposition to, as well as in the prevention and/or treatment of schizophrenia and related disorders.

### BACKGROUND OF THE INVENTION

There are an estimated 45 million people with schizophrenia in the world, with more than 33 million of them in the developing countries. In developed countries schizophrenia occurs in approximately 1% of the adult population at some point during their lives. If there is one grandparent with schizophrenia, the risk of getting the illness increases to about 3%; one parent with schizophrenia, to about 10%. When both parents have schizophrenia, the risk rises to approximately 40%. Most schizophrenia patients are never able to work. Standardized mortality ratios (SMRs) for schizophrenic patients are estimated to be two to four times higher than the general population and their life expectancy overall is 20% shorter than for the general population. The most common cause of death among schizophrenic patients is suicide (in 10% of patients) which represents a 20 times higher risk than for the general population. Deaths from heart disease and from diseases of the respiratory and digestive system are also increased among schizophrenic patients.

Schizophrenia comprises a group of psychoses with ‘positive’ and/or ‘negative’ symptoms. Positive symptoms consist of hallucinations, delusions and disorders of thought; negative symptoms include emotional flattening, lack of volition and a decrease in motor activity.

Antipsychotic medications are the most common and valuable treatments for schizophrenia. There are four main classes of antipsychotic drugs, which are commonly prescribed for schizophrenia. The first, neuroleptics, exemplified by chlorpromazine (Thorazine), has revolutionized the treatment of schizophrenic patients by reducing positive (psychotic) symptoms and preventing their recurrence. Patients receiving chlorpromazine have been able to leave mental hospitals and live in community programs or their own homes. But these drugs are far from ideal. Some 20% to 30% of patients do not respond to them at all, and others eventually relapse. These drugs were named neuroleptics because they produce serious neurological side effects, including rigidity and tremors in the arms and legs, muscle spasms, abnormal body movements, and akathisia (restless pacing and fidgeting). These side effects are so troublesome that many patients simply refuse to take the drugs. Besides, neuroleptics do not improve the so-called negative symptoms of schizophrenia and the side effects may even exacerbate these symptoms. Thus, despite the clear beneficial effects of neuroleptics, even some patients who have a good short-term response will ultimately deteriorate in overall functioning.

The well known deficiencies in the standard neuroleptics have stimulated a search for new treatments and have led to a new class of drugs termed atypical neuroleptics. The first atypical neuroleptic, Clozapine, is effective for about one third of patients who do not respond to standard neuroleptics. It seems to reduce negative as well as positive symptoms, or at least exacerbates negative symptoms less than standard neuroleptics do. Moreover, it has beneficial effects on overall functioning and may reduce the chance of suicide in schizophrenic patients. It does not produce the troubling neurological symptoms of the standard neuroleptics, or raise blood levels of the hormone prolactin, excess of which may cause menstrual irregularities and infertility in women, impotence or breast enlargement in men. Many patients who cannot tolerate standard neuroleptics have been able to take clozapine. However, clozapine has serious limitations. It was originally withdrawn from the market because it can cause agranulocytosis, a potentially lethal inability to produce white blood cells. Agranulocytosis remains a threat that requires careful monitoring and periodic blood tests. Clozapine can also cause seizures and other disturbing side effects (e.g., drowsiness, lowered blood pressure, drooling, bed-wetting, and weight gain). Thus only patients who do not respond to other drugs usually take Clozapine.

Researchers have developed a third class of antipsychotic drugs that have the virtues of clozapine without its defects. One of these drugs is risperidone (Risperdal). Early studies suggest that it is as effective as standard neuroleptic drugs for positive symptoms and may be somewhat more effective for negative symptoms. It produces more neurological side effects than clozapine but fewer than standard neuroleptics. However, it raises prolactin levels. Risperidone is now prescribed for a broad range of psychotic patients, and many clinicians seem to use it before clozapine for patients who do not respond to standard drugs, because they regard it as safer. Another new drug is Olanzapine (Zyprexa), which is at least as effective as standard drugs for positive symptoms and more effective for negative symptoms. It has few neurological side effects at ordinary clinical doses, and it does not significantly raise prolactin levels. Although it does not produce most of clozapine's most troubling side effects, including agranulocytosis, some patients taking olanzapine may become sedated or dizzy, develop dry mouth, or gain weight. In rare cases, liver function tests become transiently abnormal.

A number of biochemical abnormalities have been identified in schizophrenic patients. As a consequence, several neurotransmitter-based hypotheses have been advanced over recent years; the most popular one has been “the dopamine hypothesis,” one variant of which states that there is over-activity of the mesolimbic dopamine pathways at the level of the D₂ receptor. However, researchers have been unable to consistently find an association between various receptors of the dopaminergic system and schizophrenia.

Accordingly, molecules used for the treatment of schizophrenia have side effects and act only against the symptoms of the disease. Consequently, there is a strong need for new molecules without associated side effects that are specifically directed against targets which are involved in the causal mechanisms of such a disorder. Therefore, there is a need to identify proteins involved in such a disease, thereby providing new targets allowing new screenings for drugs, resulting in new drugs that are efficient in treatment of this serious mental disease and related disorders.

Furthermore, there is also a need for diagnostic tools. There is increasing evidence that leaving schizophrenia untreated for long periods early in course of the illness may negatively affect the outcome. However, the use of drugs is often delayed for patients experiencing a first episode of the illness. The patients may not realize that they are ill, or they may be afraid to seek help; family members sometimes hope the problem will simply disappear or cannot persuade the patient to seek treatment; clinicians may hesitate to prescribe antipsychotic medications when the diagnosis is uncertain because of potential side effects. Indeed, at the first manifestation of the disease, schizophrenia may be difficult to distinguish from, e.g., drug-related disorders and stress-related disorders. Accordingly, there is a need for new methods for detecting a susceptibility to schizophrenia and related disorders.

### SUMMARY OF THE INVENTION

The present invention now discloses novel approaches to the diagnosis and treatment of schizophrenia and related disorders, as well as for the screening of therapeutically active drugs. The invention more specifically demonstrates that alterations in the CNTFR gene are associated with the development of schizophrenia. CNTFR, and altered forms of CNTFR in particular, represent novel targets for therapeutic intervention against said disease and related pathologies.

A first aspect of this invention thus resides in the use of a CNTFR gene or polypeptide as a target for the screening of candidate drug modulators, particularly candidate drugs active against schizophrenia and related disorders.

A further aspect of this invention resides in methods of screening of compounds for therapy of schizophrenia or related disorders, comprising determining the ability of a compound to bind a CNTFR gene or polypeptide, or a fragment thereof, particularly of an allele of said gene or polypeptide that is associated with schizophrenia or a related disorder, or a fragment thereof.

A further aspect of this invention resides in methods of screening of compounds for therapy of schizophrenia or related disorders, comprising testing for modulation of the activity of a CNTFR gene or polypeptide, or a fragment thereof, particularly of an allele of said gene or polypeptide that is associated with schizophrenia, bipolar disorder or a related disorder, or a fragment thereof.

Another aspect of this invention resides in a method of assessing the presence of or predisposition to schizophrenia or a related disorder in a subject, comprising determining (in vitro or ex vivo) the presence of an alteration (e.g., a susceptibility mutation or allele) in a CNTFR gene or polypeptide in a sample from the subject, the presence of such an alteration being indicative of the presence of or predisposition to schizophrenia or a related disorder in said subject.

A further aspect of this invention relates to the use of a modulator of a CNTFR gene or polypeptide, preferably an agonist thereof, for the preparation of a medicament for treating or preventing schizophrenia or a related disorder in a subject, as well as to corresponding methods of treatment.

The invention more specifically encompasses methods of treating schizophrenia or related disorders in a subject through a modulation of CNTFR gene or polypeptide expression or activity, preferably through an activation or restoration thereof. Such treatments use, for instance, a CNTFR polypeptide, a CNTFR DNA sequence (including antisense sequences, RNAi), antibodies against CNTFR polypeptides, ligands of CNTFR or drugs that modulate, preferably mimic or stimulate, CNTFR expression or activity. The invention particularly relates to methods of treating individuals having disease-associated alleles of the CNTFR gene.

The invention further relates to the screening of alteration(s) associated with schizophrenia or related disorders in the CNTFR gene locus in patients. Such screenings are useful for diagnosing the presence, risk or predisposition to schizophrenia and related disorders, and/or for assessing the efficacy of a treatment of such disorders.

A further aspect of this invention includes nucleic acid probes and primers that allow specific detection of susceptibility markers in a CNTFR gene or RNA through selective hybridization or amplification. The invention also encompasses particular nucleic acids, vectors and recombinant cells, as well as kits or solid phase bound nucleic acids or proteins such as DNA or protein arrays or chips suitable for implementing the above detection, screening or treatment methods. In particular, the invention also discloses and encompasses markers in the CNTFR nucleic acids and polypeptides that are associated with schizophrenia and related disorders. Examples of such markers are more particularly selected from M2, M3, M4 and M9 markers as listed in Table 2, or combination(s) thereof.

The invention can be used in the diagnosis of predisposition to, detection, prevention and/or treatment of schizophrenia and related disorders in any mammalian subjects, particularly human patients.

### DETAILED DESCRIPTION OF THE INVENTION

The present invention stems from association studies conducted on different schizophrenic populations, using a number of random markers. The results of these studies, which are presented in the experimental section, show that the CNTFR gene is strongly associated with schizophrenia, and that new and validated (biallelic) markers located in said gene or corresponding RNAs are associated with schizophrenia and related disorders.

The present invention thus provides novel means and methods to identify compounds useful in the treatment of schizophrenia and related disorders. The invention further provides novel approaches to the detection, diagnosis and monitoring of schizophrenia or related disorders in a subject, as well as for genotyping of schizophrenic patients.

### Definitions

The term “schizophrenia” refers to a condition characterized as schizophrenia in the DSM-IV classification (Diagnosis and Statistical Manual of Mental Disorders, Fourth Edition, American Psychiatric Association, Washington D.C., 1994).

Schizophrenia related disorders include psychotic disorders, such as schizoaffective disorder, schizophreniform disorder, brief psychotic disorder, delusional disorder and shared psychotic disorder, as well as other mental disorders such as mood disorders and depression. Schizophrenia related disorders more particularly designate psychotic disorders as listed above.

The term “mental disorder” refers, more generally, to diseases characterized as mood disorders, psychotic disorders, anxiety disorders, childhood disorders, eating disorders, personality disorders, adjustment disorder, autistic disorder, delirium, dementia, multi-infarct dementia and Tourette's disorder in the DSM-IV classification (Diagnosis and Statistical Manual of Mental Disorders, Fourth Edition, American Psychiatric Association, Washington D.C., 1994). “Bipolar disorder” refers more specifically to a condition characterized as a Bipolar Disorder in the DSM-IV. Bipolar disorder may be bipolar I and bipolar disorder II as described in the DSM-IV. The term further includes cyclothymic disorder. Cyclothymic disorder refers to an alternation of depressive symptoms and hypomanic symptoms. The skilled artisan will recognize that there are alternative nomenclatures, posologies, and classification systems for pathologic psychological conditions and that these systems evolve with medical scientific progress.

As used in the present application, the term “CNTFR” designates the human CNTF-α receptor, as well as variants, analogs and fragments thereof. The nucleic and amino acid sequences of a CNTFR gene or polypeptide are available in the literature and may be found for instance under the following accession numbers: EMBL: M73238 (SEQ ID NO: 1 and 2, respectively); REFseqn: NM_{—}147164. The structure and signaling of the CNTFR are discussed, for instance, in Schuster et al (2003) and in Man et al (2003). These references indicate that CNTF has a neuro-protective effect in multiple sclerosis or in amyotrophic lateral sclerosis (ALS). However, no polymorphism has been described in this gene that relates to schizophrenia or related disorders, and the present invention provides the first evidence of a correlation between said gene and these diseases in human subjects.

The term “gene” shall be construed to include any type of coding nucleic acid region, including genomic DNA (gDNA), complementary DNA (cDNA), synthetic or semi-synthetic DNA, any form of corresponding RNA (e.g., mRNA), etc., as well as non coding sequences, such as introns, 5′- or 3′-untranslated sequences or regulatory sequences (e.g., promoter or enhancer), etc. The term gene particularly includes recombinant nucleic acids, i.e., any non naturally occurring nucleic acid molecule created artificially, e.g., by assembling, cutting, ligating or amplifying sequences. A gene is typically double-stranded, although other forms may be contemplated, such as single-stranded. Genes may be obtained from various sources and according to various techniques known in the art, such as by screening DNA libraries or by amplification from various natural sources. Recombinant nucleic acids may be prepared by conventional techniques, including chemical synthesis, genetic engineering, enzymatic techniques, or a combination thereof.

A fragment of a gene designates any portion of at least about 8 consecutive nucleotides of a sequence of said gene, preferably at least about 15, more preferably at least about 25 nucleotides, further preferably of at least 35, 50, 75, 100, 150, 200 or 300 nucleotides. Fragments include more particularly all possible nucleotide length between 8 and 500 nucleotides, preferably between 15 and 300, more preferably between 25 and 200.

A CNTFR polypeptide designates any protein or polypeptide encoded by a CNTFR gene as disclosed above, respectively. In this respect, the term “polypeptide” designates, within the context of this invention, a polymer of amino acids without regard to the length of the polymer; thus, peptides, oligopeptides, and proteins are included within the definition of polypeptide. In particular a CNTFR polypeptide also denotes a polypeptide, which is specific fragment of CNTFR of at least 8, 15, 20, 50, 100, 250, 300 or 350 amino acids in length. This term also does not specify or exclude post-translational or post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

Fusion proteins are useful for generating antibodies against a CNTFR polypeptide and for use in various assay systems. For example, fusion proteins can be used to identify proteins, which interact with portions of a CNTFR polypeptide. Protein affinity chromatography or library-based assays for protein-protein interactions, such as the yeast two-hybrid or phage display systems, can be used for this purpose. Such methods are well known in the art and also can be used as drug screens.

A CNTFR polypeptide fusion protein comprises two polypeptide segments fused together by means of a peptide bond. The first polypeptide segment comprises at least 25, 50, 75, 100, 150, 200, 300, 350 or 372 contiguous amino acids of SEQ ID NO: 2. The second polypeptide segment can be a full-length protein or a protein fragment. Proteins commonly used in fusion protein construction include beta-galactosidase, beta-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the CNTFR polypeptide-encoding sequence and the heterologous protein sequence, so that the CNTFR polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences for CNTFR in proper reading frame with nucleotides encoding the second polypeptide segment and expressing the DNA construct in a host cell, as is known in the art.

The term “treat” or “treating” as used herein is meant to ameliorate, alleviate symptoms, eliminate the causation of the symptoms either on a temporary or permanent basis, or to prevent or slow the appearance of symptoms of the named disorder or condition. The term “treatment” as used herein also encompasses the term “prevention of the disorder”, which is, e.g., manifested by delaying the onset of the symptoms of the disorder to a medically significant extent. Treatment of the disorder is, e.g., manifested by a decrease in the symptoms associated with the disorder or an amelioration of the reoccurrence of the symptoms of the disorder.

The terms “modulated” or “modulation” or “regulated” or “regulation” as used herein refer to both upregulation [i.e., activation or stimulation (e.g., by agonizing or potentiating)] and downregulation [i.e., inhibition or suppression (e.g., by antagonizing, decreasing or inhibiting)].

The terms “comprising”, “consisting of”, or “consisting essentially of” have distinct meanings. However, each term may be substituted for another herein to change the scope of the invention.

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe compounds comprising RNA, DNA, or RNA/DNA hybrid sequences of any length in single-stranded or duplex form. The term “nucleotide” is also used herein as a noun to refer to individual nucleotides or varieties of nucleotides, meaning a compound, or individual unit in a larger nucleic acid compound, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, for examples of analogous linking groups, purine, pyrimidines, and sugars see for example PCT publication No. WO95/04064, the disclosure of which is incorporated herein by reference. However, the polynucleotides of the invention are preferably comprised of greater than 50% conventional deoxyribose nucleotides, and most preferably greater than 90% conventional deoxyribose nucleotides. The polynucleotide sequences of the invention may be prepared by any known method, including synthetic, recombinant, ex vivo generation, or a combination thereof, as well as utilizing any purification methods known in the art.

The term “isolated” requires that the material be removed from its original environment (e.g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The term “primer” denotes a specific oligonucleotide sequence, which is complementary to a target nucleotide sequence and used to hybridize to the target nucleotide sequence. A primer serves as an initiation point for nucleotide polymerization catalyzed by either DNA polymerase, RNA polymerase or reverse transcriptase. Typical primers of this invention are single-stranded nucleic acid molecules of about 6 to 50 nucleotides in length, more preferably of about 8 to about 40 nucleotides in length, typically of about 16 to 25. The Tm is typically of about 60° C. or more. The sequence of the primer can be derived directly from the sequence of the target gene. Perfect complementarity between the primer sequence and the target gene is preferred, to ensure high specificity. However, certain mismatch may be tolerated.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified. Probes of this invention typically comprise single-stranded nucleic acids of between 10 to 1000 nucleotides in length, for instance of between 10 and 750, more preferably of between 15 and 600, typically of between 20 and 400. The sequence of the probes can be derived from the sequences of the CNTFR gene sequence. The probe may contain nucleotide substitutions and/or chemical modifications, e.g., to increase the stability of hybrids or to label the probe. Typical examples of labels include, without limitation, radioactivity, fluorescence, luminescence, etc.

The terms “complementary” or “complement thereof” are used herein to refer to the sequences of polynucleotides that are capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

As used herein, the term “non-human animal” refers to any non-human vertebrate, birds and more usually mammals, preferably primates, farm animals such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

The terms “trait” and “phenotype” are used interchangeably herein and refer to any clinically distinguishable, detectable or otherwise measurable property of an organism such as symptoms of, or susceptibility to a disease for example. Typically the terms “trait” or “phenotype” are used herein to refer to symptoms of, or susceptibility to bipolar disorder; or to refer to an individual's response to an agent acting on bipolar disorder; or to refer to symptoms of, or susceptibility to side effects to an agent acting on bipolar disorder.

As used herein, the term “allele” refers to one of the variant forms of a biallelic or multiallelic marker, differing from other forms in its nucleotide sequence. Typically the first identified allele is designated as the original allele whereas other alleles are designated as alternative alleles. Diploid organisms may be homozygous or heterozygous for an allelic form.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A polymorphism may comprise a substitution, deletion or insertion of one or more nucleotides. A single nucleotide polymorphism is a single base pair change. Typically a single nucleotide polymorphism is the replacement of one nucleotide by another nucleotide at the polymorphic site. A “single nucleotide polymorphism” (SNP) refers to a sequence polymorphism differing in a single base pair.

### Detection and Diagnosis

The present invention provides novel means and methodologies for detecting or diagnosing Schizophrenia and related disorders in a human subject. The present methods may be implemented at various development stages of said pathologies, including early, pre-symptomatic stages, and late stages, in adults, children and pre-birth. Furthermore, the invention is suited to determine the prognosis, to assess a predisposition to or a risk of development of pathology, to characterize the status of a disease or to define the most appropriate treatment regimen for a patient.

A particular object of this invention resides in a method of detecting the presence of or predisposition to schizophrenia or a related disorder in a subject, the method comprising detecting the presence of an alteration in a CNTFR gene or polypeptide in a sample from the subject, the presence of such an alteration being indicative of the presence of or predisposition to schizophrenia or a related disorder in said subject.

Another object of this invention relates to methods of assessing the response of a subject to a treatment of schizophrenia or a related disorder, the methods comprising detecting the presence of an alteration in a CNTFR gene or polypeptide in a sample from the subject, the presence of such an alteration being indicative of a responder subject.

As will be discussed below in more details, the alteration in a CNTFR gene or polypeptide may be any susceptibility marker in said gene or polypeptide, i.e., any nucleotide or amino acid alteration associated to schizophrenia or a related disease.

An alteration in the CNTFR gene may be any form of mutation(s), deletion(s), rearrangement(s) and/or insertion(s) in the coding and/or non-coding region of the gene, either isolated or in various combination(s). Mutations more specifically include point mutations. Deletions may encompass any region of two or more residues in a coding or non-coding portion of the gene. Typical deletions affect small regions, such as domains (introns) or repeated sequences or fragments of less than about 50 consecutive base pairs, although larger deletions may occur as well. Insertions may encompass the addition of one or several residues in a coding or non-coding portion of the gene. Insertions may typically comprise an addition of between 1 and 50 base pairs in the gene. Rearrangements include for instance sequence inversions. An alteration in the CNTFR gene may also be an aberrant modification of the polynucleotide sequence, such as of the methylation pattern of the genomic DNA, allelic loss of the gene or allelic gain of the gene. The alteration may be silent (i.e., create no modification in the amino acid sequence of the protein), or may result, for instance, in amino acid substitutions, frameshift mutations, stop codons, RNA splicing, e.g. the presence of a non-wild type splicing pattern of a messenger RNA transcript, or RNA or protein instability or a non-wild type level of the CNTFR polypeptide. Also, the alteration may result in the production of a polypeptide with altered function or stability, or cause a reduction or increase in protein expression levels.

Particular alterations of this invention are located in 5′ or 3′ regions of the CNTFR gene. Typical alterations are single nucleotide substitutions.

In this regard, the present invention now discloses several markers or mutations in the CNTFR gene, which are associated with schizophrenia. These mutations are reported in table 2.

Most preferred genetic alterations are disclosed in tables 2a below:

TABLE 2a

| | | | | Schizophrenia- | |
|---|---|---|---|---|---|
| | | | Poly- | associated | |
| Marker | SNP name | Location | morphism | allele | Position in sequence |
| 27-486/30 | M2 | 5′ of gene | C/T | C | 65454 in SEQ ID NO: 3 |
| 27-417/43 | M3 | 5′ of gene | A/G | G | 24120 in SEQ ID NO: 3 |
| 27-180/28 | M4 | 5′ of gene | G/C | G | 28 in SEQ ID NO: 3 |
| 27-484/27 | M9 | 3′ of gene | C/T | T | 27 in SEQ ID NO: 4 |

A preferred embodiment of the present invention comprises the detection of the presence of a marker as disclosed in Table 2 in the CNTFR gene or RNA sequence of a subject, more particularly the detection of at least one marker as disclosed in Table 2a, or any combination thereof. More specifically, the invention comprises detecting at least one marker selected from M2, M3, M4 and M9 as listed in Table 2a, the presence of a schizophrenia-associated allele being indicative of the presence, risk or predisposition to schizophrenia or a related disorder.

A preferred object of this invention is a method of detecting the presence of or predisposition to schizophrenia or a related disorder in a subject, the method comprising detecting the presence or absence of the associated allele according to table 2a of one or more of the markers M2, M3, M4 and M9 in a sample from the subject, the presence of the associated allele being indicative of the presence of or predisposition to schizophrenia or a related disorder in said subject.

Now that the association between CNTFR and schizophrenia or related diseases has been established by the inventors, it should be understood that additional susceptibility markers can be identified within said gene or polypeptide, e.g., following the methodology disclosed in the examples.

The presence of an alteration in the CNTFR gene may be detected by any technique known per se to the skilled artisan (reviewed by Kwok et al., 2003), including sequencing, pyrosequencing, selective hybridisation, selective amplification and/or mass spectrometry including matrix-assisted laser desorption/ionization time-of-flight mass spectrometry (MALDI-TOF MS) (Gut et al., 2004). In a particular embodiment, the alteration is detected by selective nucleic acid amplification using one or several specific primers, as disclosed in Table 2b below. In another particular embodiment, the alteration is detected by selective hybridization using one or several specific probes.

Further techniques include gel electrophoresis-based genotyping methods such as PCR coupled with restriction fragment length polymorphism analysis, multiplex PCR, oligonucleotide ligation assay, and minisequencing; fluorescent dye-based genotyping technologies such as oligonucleotide ligation assay, pyrosequencing, single-base extension with fluorescence detection, homogeneous solution hybridization such as TaqMan, and molecular beacon genotyping; rolling circle amplification and Invader assays as well as DNA chip-based microarray and mass spectrometry genotyping technologies (Shi et al., 2001).

Furthermore, RNA expression of altered genes can be quantified by methods known in the art such as subtractive hybridisation, quantitative PCR, TaqMan, differential display reverse transcription PCR, serial, partial sequencing of cDNAs (sequencing of expressed sequenced tags (ESTs) and serial analysis of gene expression (SAGE)), or parallel hybridization of labeled cDNAs to specific probes immobilized on a grid (macro- and microarrays and DNA chips. Particular methods include allele-specific oligonucleotide (ASO), allele-specific amplification, fluorescent in situ hybridization (FISH) Southern and Northern blot, and clamped denaturing gel electrophoresis.

Protein expression analysis methods are known in the art and include 2-dimensional gel-electrophoresis, mass spectrometry and antibody microarrays (Freeman et al., 2004 and Zhu et al., 2003).

Sequencing can be carried out using techniques well known in the art, using automatic sequencers. The sequencing may be performed on the complete gene or, more preferably, on specific domains thereof, typically those known or suspected to carry deleterious mutations or other alterations.

Amplification may be performed according to various techniques known in the art, such as by polymerase chain reaction (PCR), ligase chain reaction (LCR) and strand displacement amplification (SDA). These techniques can be performed using commercially available reagents and protocols. A preferred technique is allele-specific PCR.

Nucleic acid primers useful for amplifying sequences from the CNTFR gene are able to specifically hybridize with a portion of the CNTFR gene that either flanks or overlaps with an alteration, such as a susceptibility marker. The primer sequence overlaps with the alteration when said alteration is contained within the sequence of the CNTFR gene to which the primer hybridizes. The primer sequence flanks the alteration when the primer hybridizes with a portion of the CNTFR gene that is preferably located at a distance below 300 bp of said alteration, even more preferably below 250, 200, 150, 100, 50, 40, 30 or 20 bp from said alteration. Preferably, the primer hybridizes with a portion of the CNTFR gene that is at 5, 4, 3, 2, 1 bp distance or immediately adjacent to said alteration.

Most preferred primers are able to specifically hybridize with a portion of the CNTFR gene that either flanks or overlaps with an alteration as described in Table 2, more preferably in Table 2a. Examples of such primer sequences are disclosed in the following Table 2b (SEQ ID NO: 5 to 16). Such primers represent a particular object of the present invention.

TABLE 2b

| | | | |
|---|---|---|---|
| Oligo* | OLIGO MIS sequence | primer PCR PU | primer PCR RP |

| | | | |
|---|---|---|---|
| 27-486/30/A | GGAATCTCCCCCTCTTA | AGGCAGCTTCTAGGAATCTC | TTCTCTGCTGGGAGTATATC |
| 27-417/43/A | AATGCCTCCCCAGACCTCA | AGACCTTACTGCTCCCATAC | GGGCGCTAAGATATAGCAAC |
| 27-180/28/B | TGGGTGCCTTCTGGTTGGA | CTCCTCCTCAAACCAACTAG | TGAGAGCAGAGAAAAGGTCC |
| 27-484/27/A | GAGGTCCTTGGCTAGAAT | TTTAAACTGAGGTCCTTGGC | CAAGACTGCAAGGGAGATTC |

*A means the mis primer is sense; B means the mis primer is reverse.

Further primers of this invention are disclosed in Table 7 (SEQ ID NO: 17 to 31).

The invention also relates to the use of a nucleic acid primer or a pair of nucleic acid primers as described above in a method of detecting the presence of or predisposition to schizophrenia or a related disorder in a subject or in a method of assessing the response of a subject to a treatment of schizophrenia or a related disorder.

According to another embodiment of the present invention, the methods involve the use of a nucleic acid probe specific for a CNTFR or altered CNTFR gene or RNA, followed by the detection of the presence of a hybrid. The probe may be used in suspension or immobilized on a substrate or support. The probe is typically labelled to facilitate detection of hybrids.

In this respect, a specific object of this invention is a nucleic acid probe complementary to and specific for a region of a CNTFR gene or RNA that carries an alteration as described in Table 2, preferably in Table 2a. The probes of the present invention are, more preferably, capable of discriminating between an altered and non-altered CNTFR gene or RNA sequence, i.e., they specifically hybridise to a CNTFR gene or RNA carrying a particular alteration as described above, and essentially do not hybridise under the same hybridization conditions or with the same stability to a CNTFR gene or RNA lacking said alteration.

The invention also concerns the use of a nucleic acid probe as described above in a method of detecting the presence of or predisposition to schizophrenia or a related disorder in a subject or in a method of assessing the response of a subject to a treatment of schizophrenia or a related disorder.

The detection methods can be performed in vitro, ex vivo or in vivo, preferably in vitro or ex vivo. They are typically performed on a sample from the subject, such as any biological sample containing nucleic acids or polypeptides. Examples of such samples include fluids, tissues, cell samples, organs, biopsies, etc. Most preferred samples are blood, plasma, saliva, urine, seminal fluid, etc. The sample may be collected according to conventional techniques and used directly for diagnosis or stored. In particular, they may be obtained by non-invasive methods, such as from tissue collections. The sample may be treated prior to performing the method, in order to render or improve availability of nucleic acids or polypeptides for testing. Treatments include, for instant, lysis (e.g., mechanical, physical, chemical, etc.), centrifugation, etc. Also, the nucleic acids and/or polypeptides may be pre-purified or enriched by conventional techniques, and/or reduced in complexity. Nucleic acids and polypeptides may also be treated with enzymes or other chemical or physical treatments to produce fragments thereof. Considering the high sensitivity of the claimed methods, very few amounts of sample are sufficient to perform the assay.

The sample is typically contacted with probes or primers as disclosed above. Such contacting may be performed in any suitable device, such as a plate, tube, well, glass, etc. The contacting may performed on a substrate coated with said specific reagents, such as a nucleic acid array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids of the sample.

The finding of an altered CNTFR gene or RNA or polypeptide in the sample is indicative of the presence, predisposition or stage of progression of schizophrenia or a related disorder in the subject. Typically, one only of the above-disclosed markers is assessed, or several of them, in combination(s).

### Drug Screening

As indicated above, the present invention also provides novel targets and methods for the screening of drug candidates or leads. These screening methods include binding assays and/or functional assays, and may be performed in vitro, in cell systems or in animals.

In this regard, a particular object of this invention resides in the use of a CNTFR polypeptide as a target for screening candidate drugs for treating or preventing schizophrenia or a related disorder.

Another object of this invention resides in methods of selecting biologically active compounds, said methods comprising contacting a candidate compound with a CNTFR gene or polypeptide, and selecting compounds that bind said gene or polypeptide.

A further other object of this invention resides in methods of selecting biologically active compounds, said method comprising contacting a candidate compound with recombinant host cell expressing a CNTFR polypeptide with a candidate compound, and selecting compounds that bind said CNTFR polypeptide at the surface of said cells and/or that modulate the activity of the CNTFR polypeptide.

A “biologically active” compound denotes any compound having biological activity in a subject, preferably therapeutic activity, more preferably a neuroactive compound, and further preferably a compound that can be used for treating schizophrenia or a related disorder, or as a lead to develop drugs for treating schizophrenia or a related disorder. A “biologically active” compound preferably is a compound that modulates the activity of CNTFR.

The above methods may be conducted in vitro, using various devices and conditions, including with immobilized reagents, and may further comprise an additional step of assaying the activity of the selected compounds in a model of schizophrenia or a related disorder, such as an animal model.

A particular method of screening comprises determining the ability of a candidate compound to bind (in vitro) to the CBD (“Cytokine-Binding Domain”) domain of a CNTFR polypeptide, in particular to a region comprising the BN or BC domain of a CNTFR polypeptide.

Another particular method of screening comprises determining the ability of a candidate compound to bind to a CNTF receptor expressed at the surface of a cell, wherein said CNTF receptor comprises at least one CNTFR polypeptide. The CNTF receptor may comprise up to 3 sub-units. In a particular embodiment, the CNTF receptor comprises a CNTFR polypeptide and a β-receptor gp130 polypeptide and/or a leukaemia inhibitory factor receptor (LIFR).

Binding to the target gene or polypeptide provides an indication as to the ability of the compound to modulate the activity of said target, and thus to affect a pathway leading to schizophrenia or a related disorder in a subject. The determination of binding may be performed by various techniques, such as by labelling of the candidate compound, by competition with a labelled reference ligand, etc. For in vitro binding assays, the polypeptides may be used in essentially pure form, in suspension, immobilized on a support, or expressed in a membrane (intact cell, membrane preparation, liposome, etc.).

Modulation of activity includes, without limitation, stimulation of the surface expression of the CNTFR receptor, modulation of multimerization of said receptor (e.g., the formation of multimeric complexes with other sub-units), etc. The cells used in the assays may be any recombinant cell (i.e., any cell comprising a recombinant nucleic acid encoding a CNTFR polypeptide) or any cell that expresses an endogenous CNTFR polypeptide. Examples of such cells include, without limitation, prokaryotic cells (such as bacteria) and eukaryotic cells (such as yeast cells, mammalian cells, insect cells, plant cells, etc.). Specific examples include E. coli, Pichia pastoris, Hansenula polymorpha, Schizosaccharomyces pombe, Kluyveromyces or Saccharomyces yeasts, mammalian cell lines (e.g., Vero cells, CHO cells, 3T3 cells, COS cells, etc.) as well as primary or established mammalian cell cultures (e.g., produced from fibroblasts, embryonic cells, epithelial cells, nervous cells, adipocytes, etc.).

Preferred selected compounds are agonists of CNTFR, i.e., compounds that can bind to CNTFR and mimic the activity of an endogenous ligand thereof, such as the CNTF.

In a particular embodiment, the screening assays of the present invention use, either alone or in addition to another CNTFR sequence, an altered CNTFR gene or polypeptide, particularly a CNTFR gene or polypeptide having a mutation as listed in Table 2, more preferably a mutation as listed in Table 2a.

A further object of this invention resides in a method of selecting biologically active compounds, said method comprising contacting in vitro a test compound with a CNTFR polypeptide according to the present invention and determining the ability of said test compound to modulate the activity of said CNTFR polypeptide.

A further object of this invention resides in a method of selecting biologically active compounds, said method comprising contacting in vitro a test compound with a CNTFR gene according to the present invention and determining the ability of said test compound to modulate the expression of said CNTFR gene, preferably to stimulate expression thereof.

In another embodiment, this invention relates to a method of screening, selecting or identifying active compounds, particularly compounds active on schizophrenia or related disorders, the method comprising contacting a test compound with a recombinant host cell comprising a reporter construct, said reporter construct comprising a reporter gene under the control of a CNTFR gene promoter, and selecting the test compounds that modulate (e.g. stimulate or reduce, preferably stimulate) expression of the reporter gene.

In another embodiment, this invention relates to the use of a CNTFR polypeptide or fragment thereof, whereby the fragment is preferably a CNTFR gene-specific fragment, for isolating or generating an agonist or stimulator of the CNTFR polypeptide for the treatment of schizophrenia or a related disorder, wherein said agonist or stimulator is selected from the group consisting of:

1. a specific antibody or fragment thereof including
a) a chimeric,
b) a humanized or
c) a fully human antibody as well as

2. a bispecific or multispecific antibody,

3. a single chain (e.g. scFv) or

4. single domain antibody, or

5. a peptide- or non-peptide mimetic derived from said antibodies or

6. an antibody-mimetic such as
a) an anticalin or
b) a fibronectin-based binding molecule (e.g. trinectin or adnectin).

The generation of peptide- or non-peptide mimetics from antibodies is known in the art (Saragovi et al., 1991 and Saragovi et al., 1992).

Anticalins are also known in the art (Vogt et al., 2004). Fibronectin-based binding molecules are described in U.S. Pat. No. 6,818,418 and WO2004029224.

Furthermore, the test compound may be of various origin, nature and composition, such as any small molecule, nucleic acid, lipid, peptide, polypeptide including an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin), etc., in isolated form or in mixture or combinations.

### Pharmaceutical Compositions and Therapy

The present invention now discloses novel approaches to the treatment of schizophrenia and related disorders by modulating the activity or expression of a CNTFR gene or polypeptide. More particularly, the present invention provides the first evidence of a correlation between said gene and said diseases in human subjects, and allows the design of novel therapeutic approaches based on a modulation, preferably a stimulation or increase of a CNTFR activity.

In this regard, a particular object of this invention resides in the use of a CNTFR polypeptide, or a nucleic acid encoding the same, for the manufacture of a pharmaceutical composition for treating or preventing schizophrenia or a related disorder in a subject.

A further object of this invention resides in the use of a modulator of CNTFR for the manufacture of a pharmaceutical composition for treating or preventing schizophrenia or a related disorder in a subject. Most preferably, the modulator is an agonist or activator of a CNTFR polypeptide.

An agonist of CNTFR includes, without limitation, any compound or molecule or condition that causes activation or mimics the activity of a CNTF receptor comprising a CNTFR polypeptide, as well as any compound or molecule or condition that causes or stimulates surface expression of a functional CNTFR polypeptide. Examples of such compounds include, for instance, a wild type CNTFR polypeptide or coding nucleic acid, an activator of a CNTFR gene promoter, as well as any ligand or drug that binds a CNTF receptor comprising a CNTFR polypeptide and causes signal transduction from said receptor. Specific examples of such drugs include, for instance, CNTF, IL-6, as well as variants and derivatives thereof, and antibodies that selectively bind CNTFR, or fragments or derivatives of such antibodies having substantially the same antigen specificity.

In a preferred embodiment, the agonist is a natural ligand of CNTFR, or an antibody, such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin), that selectively binds CNTFR.

In another embodiment, the modulator is an inhibitor or antagonist of a CNTFR polypeptide.

A further object of this invention resides in a pharmaceutical composition comprising a nucleic acid encoding a CNTFR polypeptide or a vector encoding the same, and a pharmaceutically acceptable carrier or vehicle.

The above uses or compositions are particularly suited for treating or preventing schizophrenia or a related disorder in a subject presenting an alteration in the CNTFR gene or polypeptide, particularly in a subject presenting a marker as described in Table 2 above, more specifically in Table 2a.

Another object of this invention is an isolated or recombinant CNTFR gene or a fragment thereof, wherein said gene or fragment comprises a marker selected from M2, M3, M4 and M9 or a combination thereof.

The invention also relates to any vector comprising a nucleic acid as defined above. The vector may be any plasmid, phage, virus, episome, artificial chromosome, and the like. In a particular embodiment, the vector is a recombinant virus. Viral vectors may be produced from different types of viruses, including without limitation baculoviruses, retroviruses, adenoviruses, AAVs, etc., according to recombinant DNA techniques known in the art. The recombinant virus is typically replication-defective, even more preferably selected from E1- and/or E4-defective adenoviruses, Gag-, pol- and/or env-defective retroviruses and Rep- and/or Cap-defective AAVs. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. No. 5,882,877, U.S. Pat. No. 6,013,516, U.S. Pat. No. 4,861,719, U.S. Pat. No. 5,278,056 and WO94/19478.

A further aspect of this invention is a recombinant host cell comprising a vector or a nucleic acid as defined above. The recombinant cell may be any prokaryotic or eukaryotic cells as discussed above. The recombinant cell preferably expresses a recombinant CNTFR polypeptide at its surface.

A preferred embodiment of the invention is the use of an activator or agonist of CNTFR or a receptor comprising CNTFR in the preparation of a medicament for the treatment of schizophrenia or a related disorder.

A particularly preferred embodiment of the invention is the use of an activator or agonist of CNTFR or a receptor comprising CNTFR in the preparation of a medicament for the treatment of schizophrenia or a related disorder wherein the activator or agonist is an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin).

The invention also relates to a method of treating or preventing schizophrenia or a related disorder in a subject, the method comprising administering to said subject a compound that modulates, preferably that activates or mimics, expression or activity of a CNTFR gene or polypeptide as defined above.

A particular embodiment of the present invention resides in a method of treating or preventing schizophrenia or a related disorder in a subject, the method comprising (i) detecting in a sample from the subject the presence of an alteration in the CNTFR gene or polypeptide as defined above and (ii) administering to said subject an agonist of CNTFR. Preferably, said alteration is selected from the group consisting of an alteration as disclosed in Table 2, more preferably in Table 2a.

Further aspects and advantages of the present invention will be disclosed in the following experimental section, which should be regarded as illustrative and not limiting the scope of the present application.

### EXAMPLES

### 1—Description of the Schizophrenia Collections Used for the Analyses of Candidate Genes.

The association studies were performed on four different populations. One collection of samples came from Moscow, Russia (the “Rogaev” collection). The others collections came from England and were provided by the University College of London (the “UCL” collection), by the Institute of Psychiatry of London (the “IOP” collection) and by the Burnley Hospital (the “Burnley” collection).

All collections include individuals that are affected (patients or “cases”) or not affected (“controls”) by schizophrenia.

67 random markers that were unlinked and not associated with the disease were used to perform stratification study and calculate the Fst value.

TABLE 1
Description and stratification study of the four different collections

| | Institute of | | United College of | |
|---|---|---|---|---|
| | Psychiatry, | | London (UCL) | |
| Population | London (IoP) | Burnley Hospital | UCLsch | Rogaev |
| Origin | English 1 | English 2 | English 3 | Russian |
| | schizophrenic | schizophrenic | schizophrenic | schizophrenic |
| Cases | 193 (107 males) | 154 (107 males) | 180 (119 males) | 154 |
| Controls | 184 (105 males) | | 295 (142 males) | 158 |
| Stratification | Fst = −0.000174 | Fst = 0.000252 | Fst = −0.000526 | Fst = 0.000386 |
| on 67 | pvalue = 6.13E−01 | pvalue = 3.06E−01 | pvalue = 1.05E−01 | pvalue = 2.52E−01 |
| random | (NS) | (NS) | (NS) | (NS) |
| markers | | | | |

All the Fst values found for each collection indicate that these samples are genetically homogeneous, hence they are ok to be used in association analysis.

### 2—Association Studies Between Schizophrenia and the CNTFR Gene

a—Genotyping of Cases and Controls

The general strategy to perform the association studies was to individually scan the DNA samples from all individuals in each population described above in order to establish the allele frequencies of biallelic markers.

The scan procedure is based on an allele-specific primer extension reaction that allows for the differentiation of homozygous normal, heterozygous mutant and homozygous mutant samples. The reaction can be used to characterize genetic variations that include deletions, insertions and substitutions.

Briefly, a region of interest, containing the polymorphic site is amplified by PCR, using two PCR primers (Primers PU and RP). A treatment with an Alcaline Phosphatase (SAP) is applied to remove non-incorporated dNTPs. The Oligo MIS primer anneals close to the polymorphic site and is extended dependent on the polymorphism. The different extension products and the OLIGO MIS primer can be clearly differentiated in a mass spectrum.

Typically, during the microsequencing (MIS) reaction, the primer is extended by a specific number of nucleotides depending on the allele and the design of the assay. In the reaction mixture, all four nucleotides A, T, C, and G are present as either dNTPs or ddNTPs (for regular SNP assays, usually three nucleotides are present as ddNTPs and one as dNTP). The incorporation of a ddNTP terminates the extension of the MIS primer. Using a DNA polymerase that incorporates both ddNTPs and dNTPs at the same rate, the MIS reaction produces allele-specific extension products of different masses depending on the sequence analyzed. Prior to mass spectrometry, the products of the MIS reaction are desalted with a SpectroCLEAN solution and SpectroCLEAN plate (SEQUENOM), and transferred onto a SpectroCHIP microarray from SEQUENOM. The SpectroCHIP is then analyzed by the SpectroREADER (SEQUENOM) mass spectrometer.

Frequencies of every biallelic marker in each population (cases and controls) were determined by microsequencing reactions on amplified fragments obtained by genomic PCR performed on the DNA samples from each individual.

The experiments were performed as detailed below:

1.) PCR

| | Initial | Volume for 1 | Concentration in |
|---|---|---|---|
| Reagents | concentration | reaction | the final volume |
| DNA | 2.5 ng/μL | 1 μL | 0.5 ng/μL |
| Hot Star Taq Buffer | 10X | 0.5 μL | 1X |
| MgCl2 | 25 mM | 0.2 μL | 1 mM |
| dNTPs | 2.5 mM | 0.4 μL | 200 μM |
| Primer PU | 30 μM | 0.0167 μL | 100 nM |
| Primer RP | 30 μM | 0.0167 μL | 100 nM |
| Hot Star Taq | 5 U/μL | 0.02 μL | 0.02 U/μL |
| H2O molecular | 5 μL | 2.8466 μL | qsp 5 μL |
| grade qsp | | | |

95° C. 15 minutes
95° C. 20 seconds
56° C. 30 seconds
72° C. 1 minute
72° C. 3 minutes
10° C. waiting

2.) SAP PURIFICATION

| | Initial | Volume for | Concentration in |
|---|---|---|---|
| Reagents | concentration | 1 reaction | the final volume |
| ThermoSeq Buffer | 16 X | 0.1063 μL | 0.243 X |
| SAP | 12.7 U/μL | 0.0237 μL | 0.0429 U/μL |
| H2O molecular grade | 2 μL | 1.8701 μL | 7 μL |
| qsp | | | |

37° C. 20 minutes
85° C. 5 minutes
10° C. waiting

3) MICROSEQUENSING REACTION (MIS)

| | Initial | Volume for | Concentration in |
|---|---|---|---|
| Reagents | concentration | 1 réaction | the final volume |
| ThermoSeq Buffer | 16 X | 0.125 μL | 0.222 X |
| dNTP | 100 mM | 0.0045 μL | 50 μM |
| ddNTP | 10 mM | 0.045 μL | 50 μM |
| ddNTP | 10 mM | 0.045 μL | 50 μM |
| ddNTP | 10 mM | 0.045 μL | 50 μM |
| Primer MIS | 30 μM | 0.18 μL | 600 nM |
| Thermosequenase | 32 U/μL | 0.018 μL | 0.064 U/μL |
| H2O molecular grade | 2 μL | 1.5375 μL | 9 μL |
| qsp | | | |

94° C. 2 minutes
94° C. 5 seconds
52° C. 5 seconds
72° C. 5 seconds
10° C. waiting

### 4.) Cleaning—Desalting

Prior to mass spectrometry, the products of the MIS reaction are desalted with a SpectroCLEAN solution and SpectroCLEAN plate (SEQUENOM), and transferred onto a SpectroCHIP microarray from SEQUENOM.

The SpectroCHIP is then analyzed by the SpectroREADER (SEQUENOM) mass spectrometer.

The results are presented in Table 2 below.

TABLE 2
List of biallelic markers located on CNTFR

| Marker | SNP name | Location | Type | Main allele |
|---|---|---|---|---|
| 27-489/46 | M1 | 5′ of gene | C/T | T |
| 27-486/30 | M2 | 5′ of gene | C/T | |
| 27-417/43 | M3 | 5′ of gene | A/G | |
| 27-180/28 | M4 | 5′ of gene | G/C | |
| 27-783/34 | M5 | intron 1 | A/G | G |
| 27-398/28 | M6 | intron 2 | G/T | T or G |
| 13-738/40 | M7 | intron 8 | C/T | T |
| 13-579/48 | M8 | 3′ of gene | G/C | G |
| 27-484/27 | M9 | 3′ of gene | C/T | |

b—SNP Frequency Analysis

Method

Markers were analysed individually. Pearson's χ2 test (2×2) was used to compare allele frequencies between cases and controls. Data were analysed using a 3×2 χ2 test for the overall difference in genotype frequencies between cases and controls. The Exact Fisher test was performed when the conditions were not respected for the Pearson's χ2 test.

Then we calculated the difference between allelic frequencies in cases and in controls: the larger the difference in allelic frequency for a given SNP, the more probable is an association between the genomic region containing that SNP and the disorder. The “chosen” allele is the allele for which the frequency is increased in cases compared to controls.

Hardy-Weinberg equilibrium statistics were calculated separately for cases and controls data and Observed and Expected genotype frequencies were compared using a Pearson's χ2 test. A departure from Hardy-Weinberg equilibrium (HWE) in case population may indicate that a mutation had occurred, which could be responsible for increasing the risk for schizophrenia.

Results

The p-values in table 3 show the probability of association between a biallelic marker and schizophrenia. A p-value under 5e-02 suggests a significant association between the biallelic marker and schizophrenia [only the significant p-values shown].

TABLE 3
Significant p-values and associated data for SNP located within the CNTFR
gene

| | | Location on | | Allele | | | | |
|---|---|---|---|---|---|---|---|---|
| | SNP | CNTFR | Chosen | frequency | Allelic | Allelic | Genotypic p- | HWE cases |
| Collection | name | gene | allele | difference | p-value | OR | value | p-value |
| Burnley | M2 | 5′ of gene | C | 0.07 | 3.79E−02 | 1.43 | 6.77E−02 | 7.37E−01 |
| | M3 | 5′ of gene | G | 0.10 | 1.23E−02 | 1.52 | 4.48E−02 | 9.53E−01 |
| | M4 | 5′ of gene | C | 0.09 | 1.68E−02 | 1.52 | 3.31E−02 | 9.51E−01 |
| | M9 | 3′ of gene | T | 0.05 | 4.67E−02 | 1.60 | 8.83E−03 | 5.48E−01 |
| UCL | M2 | 5′ of gene | T | 0.02 | 4.76E−01 | 1.12 | 1.04E−01 | 4.22E−02 |
| | M9 | 3′ of gene | C | 0.02 | 2.98E−01 | 1.24 | 2.80E−01 | 1.07E−02 |
| Rogaev | M2 | 5′ of gene | T | 0.03 | 3.46E−01 | 1.18 | 2.01E−02 | 2.42E−03 |
| | M3 | 5′ of gene | A | 0.06 | 1.28E−01 | 1.30 | 2.78E−02 | 9.15E−03 |
| | M4 | 5′ of gene | G | 0.07 | 7.00E−02 | 1.36 | 1.51E−02 | 5.23E−03 |

| | |
|---|---|
| IOP | No significant p-values |

By estimating the allelic Odds Ratio (OR) we evaluate the probability of having the disease when carrying a given allele (=chosen [or ‘risk’] allele) compared to not carrying it.

An OR higher than 1 shows that the probability of having schizophrenia is higher when carrying the ‘risk’ allele [or genotype or haplotype] than when carrying the other ones.

The genotypic OR allows the identification of the ‘risk’ genotype(s) for an associated biallelic marker. The genotypic odds ratio was calculated and Table 4 shows the significant results.

TABLE 4
Genotypic OR for SNP located on CNTFR

| Collection | SNP name | Genotypes | Odds Ratio | Confidence Interval | p-value |
|---|---|---|---|---|---|
| Rogaev | M2 | CC vs (CT + TT) | 1.57 | 0.75-3.3 | 2.0E−01 |
| | | TT vs (CT + CC) | 1.56 | 0.99-2.44 | 5.0E−02 |
| | | (CC + TT) vs CT | 1.92 | 1.2-3.06 | 8.0E−03 |
| | M3 | AA vs (AG + GG) | 1.71 | 1.09-2.69 | 2.0E−02 |
| | | GG vs (AA + AG) | 1.14 | 0.6-2.19 | 6.0E−01 |
| | | (GG + AA) vs AG | 1.85 | 1.17-2.92 | 8.0E−03 |
| | M4 | CC vs (CG + GG) | 1.09 | 0.565-2.1 | 8.0E−01 |
| | | GG vs (CG + CC) | 1.81 | 1.15-2.84 | 9.0E−03 |
| | | (GG + CC) vs CG | 1.93 | 1.22-3.05 | 6.0E−03 |
| Burnley | M2 | CC vs (CT + TT) | 1.26 | 0.59-2.69 | 5.0E−01 |
| | | CT vs (TT + CC) | 1.58 | 1.01-2.47 | 5.0E−02 |
| | | (CC + CT) vs TT | 1.67 | 1.08-2.58 | 3.0E−02 |
| | M3 | (AG + GG) vs AA | 1.75 | 1.11-2.76 | 2.0E−02 |
| | | GG vs (AA + AG) | 1.72 | 0.9-3.31 | 1.0E−01 |
| | | AG vs (GG + AA) | 1.33 | 0.85-2.08 | 3.0E−01 |
| | M4 | CC vs (CG + GG) | 1.52 | 0.82-2.82 | 2.0E−01 |
| | | CG vs (GG + CC) | 1.4 | 0.9-2.16 | 1.0E−01 |
| | | (CG + CC) vs GG | 1.74 | 1.12-2.71 | 1.0E−02 |

Four biallelic markers located in CNTFR gene (M2, M3, M4 and M9) are associated with schizophrenia. The markers M2, M3 and M4 are highly associated in the Rogaev and Burnley collections (significant allelic and genotypic p-values or significant genotypic and HWE cases p-values).

In the Burnley collection, the risk genotypes for the M2 are CC and CT., with ‘C’ as the risk allele. For the M3 marker the risk genotypes are AG and GG, so G is the risk allele. The risk genotypes for M4 are CG and GG and G is the risk allele. For the Rogaev collection, there are no allelic associations so we cannot define a risk allele as in the Burnley collection. In the table of genotypic ORs, the genotypic associations are due to homozygous genotypes (CC+TT) for M2, (GG+AA) for M3 and (GG+CC) for M4. These differences could be explained by a difference in the specific population evolution of Burnley and Rogaev samples.

A third population [UCL] confirms the findings from the other 2 samples.

In summary, the association results of the single biallelic marker frequency analysis show that the CNTFR gene is associated with schizophrenia.

c—Haplotype Frequency Analysis

One way of increasing the statistical power of individual markers is to perform haplotype association analysis.

Haplotype analysis for association of CNTFR markers and schizophrenia was performed by estimating the frequencies of haplotypes that include the significant biallelic markers in the cases and control populations, and comparing these frequencies with a chi square test. Haplotype estimations were performed with the Expection-Maximization (EM) algorithm. More particularly, an Omnibus LR test which compares the profile of haplotype frequencies was performed.

The results are shown in the following table:

TABLE 5
Haplotype analysis of significant SNP of CNTFR gene

| | | Omnibus | | Overall | | Control | |
|---|---|---|---|---|---|---|---|
| Collection | Markers | test | Haplotype | (%) | Case (%) | (%) | OR |
| Burnley | M3-M4 | 0.012 | GC | 36.05 | 41.37 | 31.43 | 1.54 |
| | M4-M5 | 0.029 | CG | 31.23 | 36.24 | 26.98 | 1.53 |
| | M2-M3-M4 | 0.012 | CGC | 26.80 | 31.34 | 22.83 | 1.54 |
| | M3-M4-M5 | 0.019 | GCG | 30.72 | 36.07 | 26.21 | 1.59 |
| Rogaev | M4-M5 | 0.042 | GG | 65.71 | 69.84 | 61.17 | 1.47 |

This haplotype analysis further strengthens the results obtained with the single SNP analysis.

### 3—Description of the Schizophrenia Collections Used for the Analyses of Candidate Genes.

The association studies were performed on two different populations. One collection of samples came from Argentina (the “Labimo” collection). The other collection came from England and was provided by the University College of London (the “UCLbip” collection).

All collections include individuals that are affected (patients or “cases”) or not affected (“controls”) by bipolar disorder.

67 random markers that were unlinked and not associated with the disease were used to perform stratification study and calculate the Fst value.

TABLE 6
Description and stratification study of the four different collections

| | United College of London | |
|---|---|---|
| Population | (UCL) UCLbip | Labimo |
| Origin | English bipolar | Argentiniean |
| | | bipolar |
| Cases | 315 | 160 (54 males) |
| Controls | 295 (142 males) | 157 (65 males) |
| Stratification on 67 | Fst = 0.000123 | Fst = 0.000566 |
| random markers | pvalue = 3.41E−01 (NS) | pvalue = 1.68E−01 |
| | | (NS) |

The Fst values found for each collection indicate that these samples are genetically homogeneous; hence they can to be used in association analysis.

TABLE 7
Primers

| | | | |
|---|---|---|---|
| Marker | OLIGO MIS sequence | Primer PCR PU | Primer PCR RP |

| | | | |
|---|---|---|---|
| M1 | AGTTATCTGACTGGAGAAA | GACAAAGTCTCAGGGATCAC | AAATTTCCTGCAAGTGCCCC |
| M5 | CGAGGGCCCGTCTTGGG | CTACAGGCTAAGGACTCTTC | GGTAGTGACTTGTGTTCAGC |
| M6 | ACACACAGGGATGTTGACAG | GACAATGACACACAGGGATG | ATCATTGCCGCGTTTGTTGG |
| M7 | CATCAGACTGGTACTGCCTC | CATACCATTCTGCTTCCTGC | ATCTGTGAGCCTTGGGAAAC |
| M8 | AGGTATGGCTGTGAGGTTT | TGAGGAGAAGCATACACTGG | AACTTCACCAATATTTCTGGG |

### LIST OF REFERENCES

### Freeman et al., Neurochem Res. 2004 June; 29(6):1065-81

### Gut I G, Hum Mutat. 2004 May; 23(5):437-41

### Kwok P Y, Chen X, Curr Issues Mol Biol. 2003 April; 5(2): 43-60

### Man et al (JBC 278 (2003) 23285

### Saragovi et al., Science. 1991 August 16; 253(5021): 792-5

### Saragovi et al., Biotechnology (N Y). 1992 July; 10(7): 773-8

### Schuster et al (JBC 278 (2003) 9528

### Shi, Clinical Chemistry 47:2; 164-172 (2001)

### Vogt et al., Chembiochem. 2004 February 6; 5(2):191-9

Zhu H, et al., Annu Rev Biochem. 2003; 72:783-812

1-17. (canceled)

18. A method of detecting the presence of or predisposition to schizophrenia or a related disorder in a subject, the method comprising detecting the presence of a susceptibility alteration in a CNTFR gene or polypeptide in a sample from the subject, the presence of such an alteration being indicative of the presence of or predisposition to schizophrenia or a related disorder in said subject.

19. The method according to claim 18, wherein said susceptibility alteration is a single nucleotide mutation.

20. The method according to claim 18, wherein said susceptibility alteration is located within the 3′ or 5′ region of the CNTFR gene.

21. The method according to claim 20, wherein the susceptibility marker is selected from M2, M3, M4 or M9 markers as listed in Table 2, or a combination thereof.

22. The method according to claim 18, wherein the presence of an alteration in the CNTFR gene is detected by sequencing, selective hybridisation and/or selective amplification.

23. The method according to claim 22, wherein said method comprises selective amplification using one or several primers selected from SEQ ID NOs: 5 to 16.

24. A method of assessing the response of a subject to a treatment of schizophrenia or a related disorder, the method comprising detecting the presence of a susceptibility alteration in a CNTFR gene or polypeptide in a sample from the subject, the presence of such an alteration being indicative of a responder subject.

25. The method according to claim 24, wherein said susceptibility alteration is a single nucleotide mutation.

26. The method according to claim 24, wherein said susceptibility alteration is located within the 3′ or 5′ region of the CNTFR gene.

27. The method according to claim 26, wherein the susceptibility marker is selected from M2, M3, M4 or M9 markers as listed in Table 2, or a combination thereof.

28. The method according to claim 24, wherein the presence of an alteration in the CNTFR gene is detected by sequencing, selective hybridisation and/or selective amplification.

29. The method according to claim 28, wherein said method comprises selective amplification using one or several primers selected from SEQ ID NOs: 5 to 16.

30. A method of selecting biologically active compounds, said method comprising contacting a candidate compound with a CNTFR gene or polypeptide and selecting compounds that bind said gene or polypeptide.

31. The method according to claim 30, wherein said method comprises contacting a candidate compound with recombinant host cell expressing a CNTFR polypeptide and selecting compounds that bind said CNTFR polypeptide at the surface of said cells and/or that modulate the activity of said CNTFR polypeptide.

32. The method according to claim 30, further comprising a step of assaying the activity of the selected compounds in a model of schizophrenia or a related disorder.

33. The method according to claim 31, further comprising a step of assaying the activity of the selected compounds in a model of schizophrenia or a related disorder.
